(19) United States
(12) Patent Application Publication
(10) Pub. No.: US 2020/0231545 A1
(43) Pub. Date: Jul. 23, 2020

(54) CHARGED ION CHANNEL BLOCKERS AND METHODS FOR USE THEREOF

(71) Applicants: President and Fellows of Harvard College, Cambridge, MA (US); Children's Medical Center Corporation, Boston, MA (US)

(72) Inventors: Bruce P. Bean, Brookline, MA (US); Clifford J. Woolf, Newton, MA (US); Jinbo Lee, Andover, MA (US); Sooyeon Jo, Auburndale, MA (US); Ivan Tochitsky, Brookline, MA (US)

(73) Assignees: President and Fellows of Harvard College, Cambridge, MA (US); Children's Medical Center Corporation, Boston, MA (US)

(21) Appl. No.: 16/733,762

(22) Filed: Jan. 3, 2020

Related U.S. Application Data
(60) Provisional application No. 62/787,995, filed on Jan. 3, 2019.

(51) Int. Cl. C07D 211/06; A61P 11/14; A61P 23/00

(52) U.S. Cl. CPC C07D 211/06; A61P 23/00; A61P 11/14

(57) ABSTRACT

The invention provides compounds, compositions, methods and kits for the treatment of pain, itch, and neurogenic inflammation.

### RELATED APPLICATIONS

The present application claims priority under 35 U.S.C. § 119(e) to U.S. provisional application, U.S. Ser. No. 62/787,995, filed Jan. 3, 2019, which is incorporated herein by reference.

### STATEMENT AS TO FEDERALLY SPONSORED RESEARCH

This invention was made with government support under W81XWH-15-1-0480 awarded by the Department of Defense and NIH U54 HL119145 awarded by the National Institutes of Health. The government has certain rights in the invention.

### BACKGROUND OF THE INVENTION

The disclosure provides compounds useful for selective inhibition of pain and itch sensing neurons (nociceptors and pruriceptors) and in the treatment of neurogenic inflammation.

### SUMMARY OF THE INVENTION

In an aspect, the disclosure features a compound having the structure of any one of compound 1 (e.g., compound 1a, compound 1 b, or any combination thereof), compound 2 (e.g., compound 2a, compound 2b, or any combination thereof), compound 3 (e.g., compound 3a, compound 3b, or any combination thereof), compound 4 (e.g., compound 4a, compound 4b, or any combination thereof), compound 5 (e.g., compound 5a, compound 5b, or any combination thereof), and compound 6 (e.g., compound 6a, compound 6b, or any combination thereof) in Table 1, where Y⁻ is absent or an anion.

TABLE 1
Compounds (CMPDs) 1-6

| CMPD | | CMPD | | CMPD | |
|---|---|---|---|---|---|
| No. | Structure | No. | Structure | No. | Structure |
| 1 | 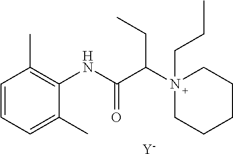 | 1a | 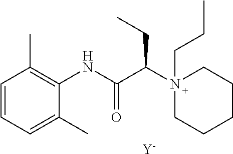 | 1b | 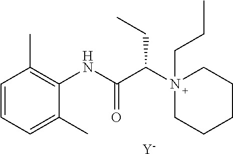 |
| 2 | 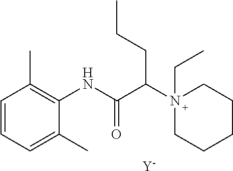 | 2a | 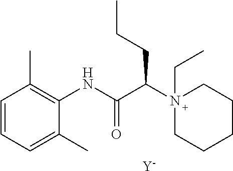 | 2b | 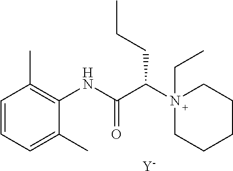 |
| 3 | 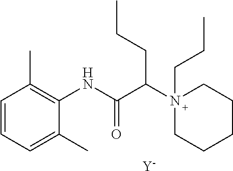 | 3a | 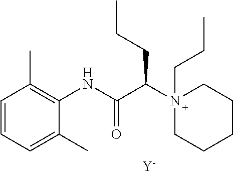 | 3b | 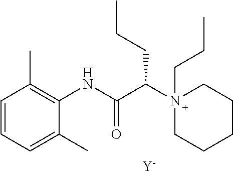 |
| 4 | 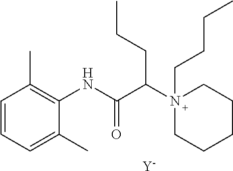 | 4a | 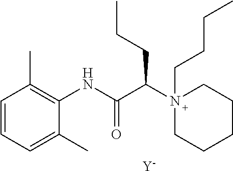 | 4b | 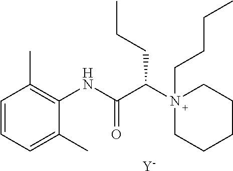 |
| 5 | 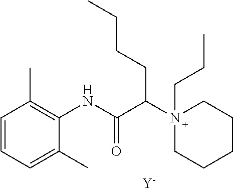 | 5a | 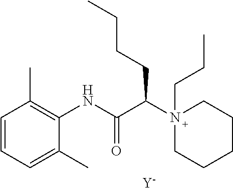 | 5b | 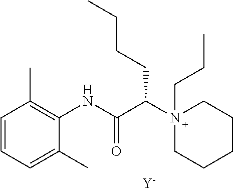 |
| 6 | 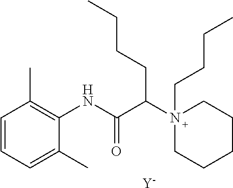 | 6a | 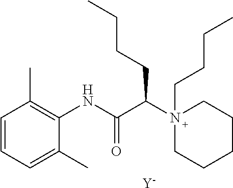 | 6b | 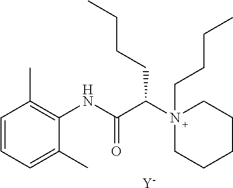 |

Y⁻ can be absent or an anion, such as an organic anion or inorganic anion. Preferably, Y⁻ can be F⁻, Ch, Br⁻, or h. More preferably, Y⁻ can be Ch or Br⁻. The disclosure features a pharmaceutical composition including any one of the foregoing compounds, and salts thereof and a pharmaceutically acceptable excipient. The composition can be formulated for oral, intravenous, intramuscular, rectal, cutaneous, subcutaneous, topical, transdermal, sublingual, pulmonary, nasal, inhalation, vaginal, intrathecal, epidural, or ocular administration, preferably for topical or inhalation administration, such as in the form of an aerosol.

The disclosure features a method for treating pain in a patient comprising administering to the patient one or more of the foregoing compounds or a pharmaceutical composition thereof. The pain can be selected from the group consisting of neuropathic pain, inflammatory pain, nociceptive pain, pain due to infections, pain due to trauma, surgical pain, and procedural pain.

The disclosure features a method for treating a neurogenic inflammatory disorder, or a symptom associated therewith, in a patient, comprising administering to the patient one or more of the foregoing compounds or a pharmaceutical composition thereof. The neurogenic inflammatory disorder can be selected from the group consisting of allergic inflammation, asthma, chronic cough, conjunctivitis, rhinitis, psoriasis, inflammatory bowel disease, interstitial cystitis, and atopic dermatitis.

The disclosure features a method for treating cough comprising administering to the patient one or more of the foregoing compounds or a pharmaceutical composition thereof. The cough can be a dry cough, wet cough, croup cough, whooping cough, chronic cough or acute cough. In particular, the disclosure provides a method for treating acute or chronic cough comprising administering to the patient one or more of the foregoing compounds or a pharmaceutical composition thereof.

The disclosure features a method for treating itch in a patient comprising administering to the patient one or more of the foregoing compounds or a pharmaceutical composition thereof. The itch can be pruritis.

In one aspect, the disclosure features a kit including one or more of the foregoing compounds or a pharmaceutical composition thereof. The kit can include a package insert instructing a user of the kit to administer the compound or pharmaceutical composition to a patient in accordance with any of the foregoing methods.

Without being bound by theory, the invention involves transient receptor potential ion channels (TRP channel-forming receptors). The TRP channel-forming receptor, such as TRPA1 or TRPV1, can be activated by an exogenous or endogenous agonist. The compounds may enter nociceptors or pruriceptors through the TRPA1 or TRPV1 receptor when the receptor is activated and inhibits voltage-gated sodium channels.

### BRIEF DESCRIPTION OF THE DRAWINGS

FIG. 1 shows the time course of peak sodium current as a function of time for cells dialyzed with either 10 micromolar QX-314 (open triangles) or 10 micromolar compound 1 (closed circles) or with control internal solution with no added compound (open circles) and stimulated with a series of 30-msec depolarizations to −20 mV from a holding potential of −100 mV. To induce use-dependent block, the depolarizations were delivered by series of increasing rates: 0.05 Hz for 1-min, 0.33 Hz for 1-min, 1 Hz for 1-min, 3 Hz for 1-min, 5 Hz for 30 seconds, 10 Hz for 30 seconds, with 1 minute rest between each series of pulses. After the series of pulses to induce use-dependent block, the time course of recovery was followed using pulses delivered at 0.1 Hz (2-min) and 0.05 Hz (1-min). Peak sodium current was plotted as a function of experimental time (1-min per division on the time axis).

FIG. 2 shows the time course of peak sodium current as a function of time for cells dialyzed with either 10 micromolar QX-314 (open triangles) or 10 micromolar compound 2 (closed circles) or with control internal solution with no added compound (open circles) and stimulated with a series of 30-msec depolarizations to −20 mV from a holding potential of −100 mV. To induce use-dependent block, the depolarizations were delivered by series of increasing rates: 0.05 Hz for 1-min, 0.33 Hz for 1-min, 1 Hz for 1-min, 3 Hz for 1-min, 5 Hz for 30 seconds, 10 Hz for 30 seconds, with 1 minute rest between each series of pulses. After the series of pulses to induce use-dependent block, the time course of recovery was followed using pulses delivered at 0.1 Hz (2-min) and 0.05 Hz (1-min). Peak sodium current was plotted as a function of experimental time (1-min per division on the time axis).

FIG. 3 demonstrates the Naᵥ1.5 sodium channel activity measured in human embryonic kidney cells via the thallium flux assay in the presence of compound 1. Compound 1 blocks Naᵥ1.5 channel function in a dose dependent manner starting at 10 millimolar extracellular concentrations. No apparent Naᵥ subtype selectivity is observed.

FIG. 4 demonstrates the Naᵥ1.7 sodium channel activity measured in human embryonic kidney cells via the thallium flux assay in the presence of compound 1. Compound 1 blocks Naᵥ1.7 channel function in a dose dependent manner starting at 10 millimolar extracellular concentrations. No apparent Naᵥ subtype selectivity is observed.

FIG. 5 shows patch clamp electrophysiological recordings of endogenous Naᵥ currents from a TRPV1 positive mouse DRG neuron treated with capsaicin alone (bottom curve) and one treated with capsaicin+compound 1 (top curve) after which the compounds were washed off prior to recording. The Naᵥ current is significantly reduced in the compound 1-treated neuron.

FIG. 6 shows that treatment of DRG neurons with capsaicin+compound 1 or QX-314 produces robust block of endogenous mouse DRG Naᵥ currents.

FIG. 7 shows the reduction in a citric acid nebulization induced cough in ovalbumin-sensitized guinea pigs with antigen-induced pulmonary inflammation produced by inhaled treatment one hour before the citric acid challenge with the charged voltage-gated sodium channel blocker compounds 1 and 2.

### DETAILED DESCRIPTION

The disclosure features quaternary ammonium compounds having the structure of any one of compound 1 (e.g., compound 1a, compound 1 b, or any combination thereof), compound 2 (e.g., compound 2a, compound 2b, or any combination thereof), compound 3 (e.g., compound 3a, compound 3b, or any combination thereof), compound 4 (e.g., compound 4a, compound 4b, or any combination thereof), compound 5 (e.g., compound 5a, compound 5b, or any combination thereof), and compound 6 (e.g., compound 6a, compound 6b, or any combination thereof). Y⁻ is absent or counterion.

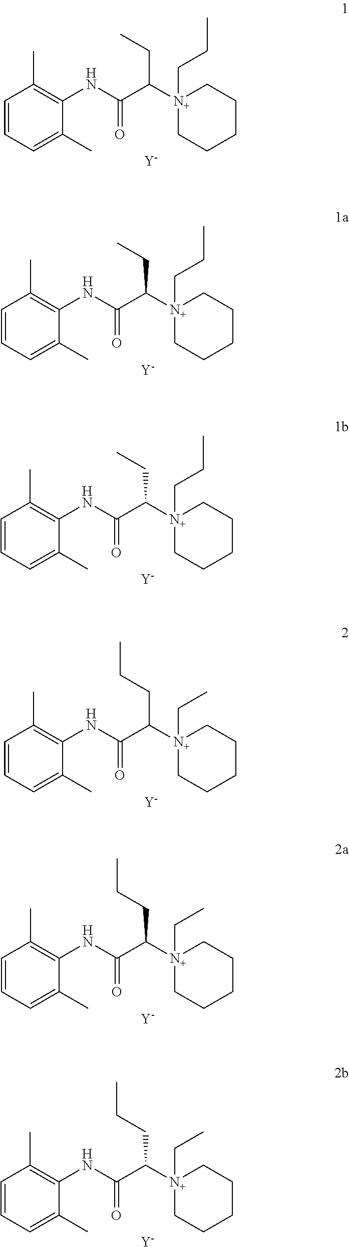

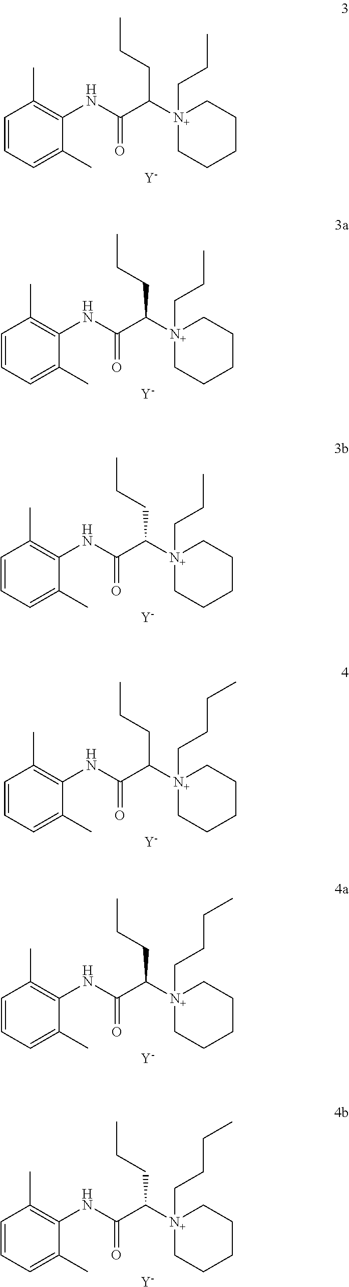

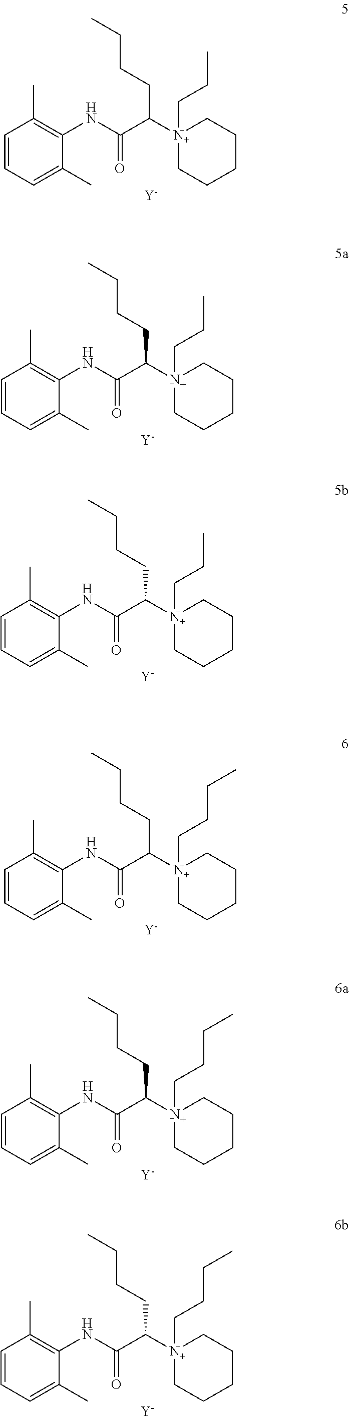

Compound 1 (e.g., compound 1a, compound 1 b, or any combination thereof) and compound 2 (e.g., compound 2a, compound 2b, or any combination thereof) are capable of passing through open TRP channel-forming receptors that are expressed on nociceptors and/or pruriceptors but not on motor neurons when applied inside cells. Because the ion channel blocking compound of the present invention is positively charged, it is not membrane-permeant and thus cannot enter cells that do not express TRP channel-forming receptors. Since TRP channel-forming receptors are often more active in tissue conditions associated with pain (such as inflammation) due to release of endogenous ligands or activation by thermal stimuli, the ion channel blocker of the invention can be used alone to selectively target activated nociceptors in order to effectively treat (e.g., eliminate or alleviate) pain, itch, or neurogenic inflammation. The ion channel blocker of the invention can also be used in combination with one or more exogenous TRP channel-forming receptor agonists to selectively target nociceptors in order to effectively treat (e.g., eliminate or alleviate) pain, itch, or neurogenic inflammation.

Voltage-dependent ion channels in pain-sensing neurons are currently of great interest in developing drugs to treat pain. Blocking voltage-dependent sodium channels in pain-sensing neurons can block pain signals by interrupting initiation and transmission of the action potential, and blocking calcium channels can prevent neurotransmission of the pain signal to the second order neuron in the spinal cord. Moreover, blocking voltage-dependent sodium channels in nociceptors can reduce or eliminate neurogenic inflammation by preventing activation of nociceptor peripheral terminals and the release thereof pro-inflammatory chemicals.

Heretofore, a limitation in designing small organic molecules that block sodium channels or calcium channels is that they must be active when applied externally to the target cell. The vast majority of such externally applied molecules are hydrophobic and can pass through membranes. Because of this, they will enter all cells and thus have no selectivity for affecting only nociceptors.

The inhibitor of the present invention is membrane-impermeant and is effective when present inside the nociceptor cell, and thus must pass through the cell membrane via a channel or receptor, such as a transient receptor potential ion channel (TRP channels, e.g., TRPAV1, TRPA1, and P2X(2/3)) to produce an effect. Under normal circumstances, most TRP channels in nociceptors are not active but require a noxious thermal, mechanical, or chemical stimulus to activate them. For example, TRP channels in nociceptors can be activated by an exogenous TRP ligand (i.e. TRP agonist) such as capsaicin, which opens the TRPV1 channel. Thus, one approach to selectively targeting nociceptors is to co-administer the membrane-impermeant ion channel inhibitor with an exogenous TRP ligand that permits passage of the inhibitor through the TRP channel into the cell. In addition to capsaicin, the exogenous TRP ligand can also be another capsaicinoid, mustard oil, or lidocaine. In another example, TRP channels may be active in response to exogenous irritant activators such as inhaled acrolein from smoke or chemical warfare agents such as tear gas.

Under certain circumstances, TRP channels can be activated in the absence of exogenous TRP agonists/ligands by endogenous inflammatory activators that are generated by tissue damage, infection, autoimmunity, atopy, ischemia, hypoxia, cellular stress, immune cell activation, immune mediator production, and oxidative stress. Under such conditions, endogenous molecules (e.g., protons, lipids, and reactive oxygen species) can activate TRP channels expressed on nociceptors, allowing membrane-impermeant, voltage-gated ion channel blockers to gain access to the inside of the nociceptor through the endogenously-activated TRP channels. Endogenous inflammatory activators of TRP channels include, for example, prostaglandins, nitric oxide (NO), peroxide (H₂O₂), cysteine-reactive inflammatory mediators like 4-hydroxynonenal, endogenous alkenyl aldehydes, endocannabinoids, and immune mediators (e.g., interleukin 1 (IL-1), nerve growth factor (NGF), and bradykinin, whose receptors are coupled to TRP channels).

### Definitions

By “biologically active” is meant that a molecule, including biological molecules such as nucleic acids, peptides, polypeptides, and proteins, exerts a physical or chemical activity on itself or other molecule. For example, a “biologically active” molecule may possess, e.g., enzymatic activity, protein binding activity (e.g., antibody interactions), or cytotoxic activities (e.g., anti-cancer properties). Biologically active agents that can be used in the methods and kits described herein include, without limitation, TRP1A receptor agonists, TRPV1 receptor agonists, P2X receptor agonists, NSAIDS, glucocorticoids, narcotics, anti-proliferative and immune modulatory agents, an antibody or antibody fragment, an antibiotic, a polynucleotide, a polypeptide, a protein, an anti-cancer agent, a growth factor, and a vaccine.

By “inflammation” is meant any types of inflammation, such those caused by the immune system (immune-mediated inflammation) and by the nervous system (neurogenic inflammation), and any symptom of inflammation, including redness, heat, swelling, pain, and/or loss of function.

By “neurogenic inflammation” is meant any type of inflammation mediated or contributed to by neurons (e.g. nociceptors) or any other component of the central or peripheral nervous system.

The term “pain” is used herein in the broadest sense and refers to all types of pain, including acute and chronic pain, such as nociceptive pain, e.g. somatic pain and visceral pain; inflammatory pain, dysfunctional pain, idiopathic pain, neuropathic pain, e.g., centrally generated pain and peripherally generated pain, migraine, and cancer pain.

The term “nociceptive pain” is used to include all pain caused by noxious stimuli that threaten to or actually injure body tissues, including, without limitation, by a cut, bruise, bone fracture, crush injury, burn, and the like. Pain receptors for tissue injury (nociceptors) are located mostly in the skin, musculoskeletal system, or internal organs.

The term “somatic pain” is used to refer to pain arising from bone, joint, muscle, skin, or connective tissue. This type of pain is typically well localized.

The term “visceral pain” is used herein to refer to pain arising from visceral organs, such as the respiratory, gastrointestinal tract and pancreas, the urinary tract and reproductive organs. Visceral pain includes pain caused by tumor involvement of the organ capsule. Another type of visceral pain, which is typically caused by obstruction of hollow viscus, is characterized by intermittent cramping and poorly localized pain. Visceral pain may be associated with inflammation as in cystitis or reflux esophagitis.

The term “inflammatory pain” includes pain associates with active inflammation that may be caused by trauma, surgery, infection and autoimmune diseases.

The term “neuropathic pain” is used herein to refer to pain originating from abnormal processing of sensory input by the peripheral or central nervous system consequent on a lesion to these systems.

The term “procedural pain” refers to pain arising from a medical, dental or surgical procedure wherein the procedure is usually planned or associated with acute trauma.

The term “itch” is used herein in the broadest sense and refers to all types of itching and stinging sensations localized and generalized, acute intermittent and persistent. The itch may be idiopathic, allergic, metabolic, infectious, drug-induced, due to liver, kidney disease, or cancer. “Pruritus” is severe itching.

By “patient” is meant any animal. In one embodiment, the patient is a human. Other animals that can be treated using the methods, compositions, and kits of the invention include but are not limited to non-human primates (e.g., monkeys, gorillas, chimpanzees), domesticated animals (e.g., horses, pigs, goats, rabbits, sheep, cattle, llamas), and companion animals (e.g., guinea pigs, rats, mice, lizards, snakes, dogs, cats, fish, hamsters, and birds).

By “low molecular weight” is meant less than about 500 Daltons.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compound of the invention, or separately by reacting the free base function with a suitable acid. Representative acid addition salts include but are not limited to acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like.

By “charged moiety” is meant a moiety which gains a proton at physiological pH thereby becoming positively charged (e.g., ammonium, guanidinium, or amidinium) or a moiety that includes a net formal positive charge without protonation (e.g., quaternary ammonium). The charged moiety may be either permanently charged or transiently charged.

As used herein, the term “parent” refers to a channel blocking compound which can be modified by quaternization or guanylation of an amine nitrogen atom present in the parent compound. The quaternized and guanylated compound is a derivative of the parent compound. The compound can be administered either as a salt (i.e., an acid addition salt) or in its uncharged base form, which undergoes protonation in situ to form a charged moiety.

By “therapeutically effective amount” means an amount sufficient to produce a desired result, for example, the reduction or elimination of pain, itch, or neurogenic inflammation in a patient (e.g., a human) suffering from a condition, disease, or illness that is caused wholly or in part by neurogenic inflammation (e.g. asthma, arthritis, colitis, contact dermatitis, diabetes, eczema, cystitis, gastritis, migraine headache, psoriasis, rhinitis, rosacea, or sunburn).

“Solvates” means solvent addition forms that contain either stoichiometric or nonstoichiometric amounts of solvent.

The compound of the present invention, including salts of the compound, can exist in unsolvated forms as well as solvated forms, including hydrated forms and unhydrated forms. In general, the solvated forms are equivalent to unsolvated forms and are encompassed within the scope of the present invention. Nonlimiting examples of hydrates include monohydrates, dihydrates, etc. Nonlimiting examples of solvates include ethanol solvates, acetone solvates, etc.

The compound of the invention may exist in multiple crystalline or amorphous forms. In general, all physical forms are equivalent for uses contemplated by the present invention and are intended to be within the scope of the invention.

In one embodiment of the present invention is a compound represented by compounds 1-6 as described above.

A composition of the invention can comprise a compound of the invention as a racemic mixture, a pure enantiomer, or an excess of one enantiomer over the other. For example, the composition can comprise the compound in an enantiomeric excess of at least 5, 10, 20, 30, 40, 50, 60, 70, 80 or 90%. In one embodiment, the enantiomeric excess is at least 95%.

The compound of the invention includes all enantiomers which may be defined, in terms of absolute stereochemistry, as (R)- or (S)-, as well as their racemic and optically pure forms, and is not limited to those described herein in any of their pharmaceutically acceptable forms, including enantiomers, salts, solvates, polymorphs, solvatomorphs, hydrates, anhydrous and other crystalline forms and combinations thereof. Likewise, all tautomeric forms are intended to be included.

Preferably, a pharmaceutical composition comprises the novel compound of the invention as an R enantiomer in substantially pure form; or, a pharmaceutical composition comprises the novel compound of the invention as an S enantiomer in substantially pure form; or, a pharmaceutical composition comprises the novel compound of the invention as enantiomeric mixtures which contain an excess of the R enantiomer or an excess of the S enantiomer. It is particularly preferred that the pharmaceutical contains the compound of the invention which is a substantially pure optical isomer. For the avoidance of doubt, the novel compound of the invention can, if desired, be used in the form of solvates.

### Exogenous TRP Channel-Forming Receptor Agonists

TRPV1 agonists are biologically active agents which can be employed in the methods and kits of the invention and include, but are not limited to, any that activates TRPV1 receptors on nociceptors and allows for entry of at least one inhibitor of voltage-gated ion channels. A suitable TRPV1 agonist is capsaicin or another capsaicinoids, which are members of the vanilloid family of molecules. Naturally occurring capsaicinoids are capsaicin itself, di hydrocapsaicin, nordihydrocapsaicin, homodihydrocapsaicin, homocapsaicin, and nonivamide. Other suitable capsaicinoids and capsaicinoid analogs and derivatives for use in the compositions and methods of the present invention include naturally occurring and synthetic capsaicin derivatives and analogs including, e.g., vanilloids (e.g., N-vanillyl-alkanedienamides, N-vanillyl-alkanedienyls, and N-vanillyl-cis-monounsaturated alkenamides), capsiate, dihydrocapsiate, nordihydrocapsiate and other capsinoids, capsiconiate, dihydrocapsiconiate and other coniferyl esters, capsiconinoid, resiniferatoxin, tinyatoxin, civamide, N-phenylmethylalkenamide capsaicin derivatives, olvanil, N-[(4-(2-aminoethoxy)-3-methoxyphenyOrnethyl]-9Z-octa-decanamide, N-oleyl-homovanillamide, triprenyl phenols (e.g., scutigeral), gingerols, piperines, shogaols, guaiacol, eugenol, zingerone, nuvanil, NE-19550, NE-21610, and NE-28345. Additional capsaicinoids, their structures, and methods of their manufacture are described in U.S. Pat. Nos. 7,446,226 and 7,429,673, which are hereby incorporated by reference.

Additional suitable TRPV1 agonists include but are not limited to eugenol, arvanil (N-arachidonoylvanillamine), anandamide, 2-aminoethoxydiphenyl borate (2APB), AM404, resiniferatoxin, phorbol 12-phenylacetate 13-acetate 20-homovanillate (PPAHV), olvanil (NE 19550), OLDA (N-oleoyldopamine), N-arachidonyldopamine (NADA), 6′-iodoresiniferatoxin (6′-IRTX), C18 N-acylethanolamines, lipoxygenase derivatives such as 12-hydroperoxyeicosatetraenoic acid, inhibitor cysteine knot (ICK) peptides (vanillotoxins), piperine, MSK195 (N-[2-(3,4-dimethylbenzyI)-3-(pivaloyloxy)propyl]-2-[4-(2-aminoethoxy)-3-methoxyphenyl]acetamide), JYL79 (N-[2-(3,4-dimethylbenzyl)-3-(pivaloyloxy)propyl]-N′-(4-hydroxy-3-methoxybenzyl)thiourea), hydroxy-alpha-sanshool, 2-aminoethoxydiphenyl borate, 10-shogaol, oleylgingerol, oleylshogaol, and SU200 (N-(4-tert-butylbenzyl)-N′-(4-hydroxy-3-methoxybenzyl)thiourea). Still other TRPV1 agonists include amylocaine, articaine, benzocaine, bupivacaine, carbocaine, carticaine, chloroprocaine, cyclomethycaine, dibucaine (cinchocaine), dimethocaine (larocaine), etidocaine, hexylcaine, levobupivacaine, lidocaine, mepivacaine, meprylcaine (oracaine), metabutoxycaine, piperocaine, prilocaine, procaine (novacaine), proparacaine, propoxycaine, risocaine, ropivacaine, tetracaine (amethocaine), and trimecaine.

Other biologically active agents which can be employed in the methods, compositions, and kits of the invention include any agonists that activate TRP1A receptors on nociceptors or pruriceptors and allows for entry of at least one inhibitor of voltage-gated ion channels. Suitable TRP1A agonists include but are not limited to cinnamaldehyde, allyl-isothiocynanate (mustard oil), diallyl disulfide, icilin, cinnamon oil, wintergreen oil, clove oil, acrolein, hydroxy-alpha-sanshool, 2-aminoethoxydiphenyl borate, 4-hydroxynonenal, methyl p-hydroxybenzoate, and 3′-carbamoylbiphenyl-3-yl cyclohexylcarbamate (URB597).

P2X agonists are biologically active agents that can be employed in the methods, compositions, and kits of the invention, and include any that activates P2X receptors on nociceptors or pruriceptors and allows for entry of at least one inhibitor of voltage-gated ion channels. Suitable P2X agonists include but are not limited to 2-methylthio-ATP, 2′ and 3′-O-(4-benzoylbenzoyl)-ATP, and ATPS′-O-(3-thiotriphosphate).

### Additional Agents

If desired, one or more additional biologically active agents typically used to treat neurogenic inflammation may be used in combination with a composition of the invention described herein. The biologically active agents include, but are not limited to, TRP1A receptor agonists, TRPV1 receptor agonists, P2X receptor agonists, acetaminophen, NSAIDS, glucocorticoids, narcotics, tricyclic antidepressants, amine transporter inhibitors, anticonvulsants, anti-proliferative and immune modulatory agents, an antibody or antibody fragment, an antibiotic, a polynucleotide, a polypeptide, a protein, an anti-cancer agent, a growth factor, and a vaccine.

The biologically active agents can be administered prior to, concurrent with, or following administration of a composition of the invention, using any formulation, dosing, or administration known in the art that is therapeutically effective.

Non-steroidal anti-inflammatory drugs (NSAIDs) that can be administered to a patient (e.g., a human) suffering from neurogenic inflammation in combination with a composition of the invention include, but are not limited to, acetylsalicylic acid, amoxiprin, benorylate, benorilate, choline magnesium salicylate, diflunisal, ethenzamide, faislamine, methyl salicylate, magnesium salicylate, salicyl salicylate, salicylamide, diclofenac, aceclofenac, acemethacin, alclofenac, bromfenac, etodolac, indometacin, nabumetone, oxametacin, proglumetacin, sulindac, tolmetin, ibuprofen, alminoprofen, benoxaprofen, carprofen, dexibuprofen, dexketoprofen, fenbufen, fenoprofen, flunoxaprofen, flurbiprofen, ibuproxam, indoprofen, ketoprofen, ketorolac, loxoprofen, naproxen, oxaprozin, pirprofen, suprofen, tiaprofenic acid, mefenamic acid, flufenamic acid, meclofenamic acid, tolfenamic acid, phenylbutazone, ampyrone, azapropazone, clofezone, kebuzone, metamizole, mofebutazone, oxyphenbutazone, phenazone, sulfinpyrazone, piroxicam, droxicam, lornoxicam, meloxicam, tenoxicam, and the COX-2 inhibitors celecoxib, etoricoxib, lumiracoxib, parecoxib, rofecoxib, valdecoxib, and pharmaceutically acceptable salts thereof.

Glucocorticoids that can be administered to a patient (e.g., a human) suffering from neurogenic inflammation in combination with a composition of the invention include, but are not limited to, hydrocortisone, cortisone acetate, prednisone, prednisolone, methylprednisolone, dexamethasone, betamethasone, triamcinolone, beclometasone, fludrocortisone acetate, deoxycorticosterone acetate, aldosterone, and pharmaceutically acceptable salts thereof.

Narcotics that can be administered to a patient (e.g., a human) suffering from neurogenic inflammation in combination with a composition of the invention include, but are not limited, to tramadol, hydrocodone, oxycodone, morphine, and pharmaceutically acceptable salts thereof.

Antiproliferative and immune modulatory agents that can be administered to a patient (e.g., a human) suffering from neurogenic inflammation in combination with a composition of the invention include, but are not limited to, alkylating agents, platinum agents, antimetabolites, topoisomerase inhibitors, dihydrofolate reductase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF-alpha agonists, TNF-alpha antagonists or scavengers, interleukin 1 (IL-1) antagonists or scavengers, endothelin A receptor antagonists, retinoic acid receptor agonists, hormonal agents, antihormonal agents, photodynamic agents, and tyrosine kinase inhibitors.

### Formulation of Compositions

The administration of the compound of the invention may be by any suitable means that results in the reduction of perceived pain sensation at the target region. The compound of the invention may be contained in any appropriate amount in any suitable carrier substance, and are generally present in amounts totaling 1-95% by weight of the total weight of the composition. The composition may be provided in a dosage form that is suitable for oral, parenteral (e.g., intravenous, intramuscular), rectal, cutaneous, subcutaneous, topical, transdermal, sublingual, nasal, vaginal, intrathecal, epidural, or ocular administration, or by injection, inhalation, or direct contact with the nasal or oral mucosa. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

The compound of the invention may be formulated in a variety of ways that are known in the art. For example, the compound of the invention and a biologically active agent as defined herein may be formulated together or separately. Desirably, the compound of the invention and the biologically active agent are formulated together for their simultaneous or near simultaneous administration. In another embodiment, two or more biologically active agents may be formulated together with the compound of the invention, or separately.

The individually or separately formulated agents can be packaged together as a kit. Non-limiting examples include but are not limited to kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions.

The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

### Controlled Release Formulations

The compound of the invention, alone or in combination with one or more of the biologically active agents as described herein, can be formulated for controlled release (e.g., sustained or measured) administration, as described in U.S. Patent Application Publication Nos. 2003/0152637 and 2005/0025765, each incorporated herein by reference. For example, the compound of the invention, alone or in combination with one or more of the biologically active agents as described herein, can be incorporated into a capsule or tablet that is administered to the site of inflammation.

Any pharmaceutically acceptable vehicle or formulation suitable for local infiltration or injection into a site to be treated (e.g., a painful surgical incision, wound, or joint), that is able to provide a sustained release of the compound of the invention, alone or in combination with one or more of the biologically active agents as described herein, may be employed to provide for prolonged elimination or alleviation of inflammation, as needed. Slow release formulations known in the art include specially coated pellets, polymer formulations or matrices for surgical insertion or as sustained release microparticles, e.g., microspheres or microcapsules, for implantation, insertion, infusion or injection, wherein the slow release of the active medicament is brought about through sustained or controlled diffusion out of the matrix and/or selective breakdown of the coating of the preparation or selective breakdown of a polymer matrix. Other formulations or vehicles for sustained or immediate delivery of an agent to a preferred localized site in a patient include, e.g., suspensions, emulsions, gels, liposomes and any other suitable art known delivery vehicle or formulation acceptable for subcutaneous or intramuscular administration.

A wide variety of biocompatible materials may be utilized as a controlled release carrier to provide the controlled release of the compound of the invention, alone or in combination with one or more biologically active agents, as described herein. Any pharmaceutically acceptable biocompatible polymer known to those skilled in the art may be utilized. It is preferred that the biocompatible controlled release material degrade in vivo within about one year, preferably within about 3 months, more preferably within about two months. More preferably, the controlled release material will degrade significantly within one to three months, with at least 50% of the material degrading into non-toxic residues, which are removed by the body, and 100% of the compound of the invention being released within a time period within about two weeks, preferably within about 2 days to about 7 days. A degradable controlled release material should preferably degrade by hydrolysis, either by surface erosion or bulk erosion, so that release is not only sustained but also provides desirable release rates. However, the pharmacokinetic release profile of these formulations may be first order, zero order, bi- or multi-phasic, to provide the desired reversible local anesthetic effect over the desired time period.

Suitable biocompatible polymers can be utilized as the controlled release material. The polymeric material may comprise biocompatible, biodegradable polymers, and in certain preferred embodiments is preferably a copolymer of lactic and glycolic acid. Preferred controlled release materials which are useful in the formulations of the invention include the polyanhydrides, polyesters, co-polymers of lactic acid and glycolic acid (preferably wherein the weight ratio of lactic acid to glycolic acid is no more than 4:1 i.e., 80% or less lactic acid to 20% or more glycolic acid by weight)) and polyorthoesters containing a catalyst or degradation enhancing compound, for example, containing at least 1% by weight anhydride catalyst such as maleic anhydride. Examples of polyesters include polylactic acid, polyglycolic acid and polylactic acid-polyglycolic acid copolymers. Other useful polymers include protein polymers such as collagen, gelatin, fibrin and fibrinogen and polysaccharides such as hyaluronic acid.

The polymeric material may be prepared by any method known to those skilled in the art. For example, where the polymeric material is comprised of a copolymer of lactic and glycolic acid, this copolymer may be prepared by the procedure set forth in U.S. Pat. No. 4,293,539, incorporated herein by reference. Alternatively, copolymers of lactic and glycolic acid may be prepared by any other procedure known to those skilled in the art. Other useful polymers include polylactides, polyglycolides, polyanhydrides, polyorthoesters, polycaprolactones, polyphosphazenes, polyphosphoesters, polysaccharides, proteinaceous polymers, soluble derivatives of polysaccharides, soluble derivatives of proteinaceous polymers, polypeptides, polyesters, and polyorthoesters or mixtures or blends of any of these. Pharmaceutically acceptable polyanhydrides which are useful in the present invention have a water-labile anhydride linkage. The rate of drug release can be controlled by the particular polyanhydride polymer utilized and its molecular weight. The polysaccharides may be poly-1,4-glucans, e.g., starch glycogen, amylose, amylopectin, and mixtures thereof. The biodegradable hydrophilic or hydrophobic polymer may be a water-soluble derivative of a poly-1,4-glucan, including hydrolyzed amylopectin, hydroxyalkyl derivatives of hydrolyzed amylopectin such as hydroxyethyl starch (HES), hydroxyethyl amylose, dialdehyde starch, and the like. The polyanhydride polymer may be branched or linear. Examples of polymers which are useful in the present invention include (in addition to homopolymers and copolymers of poly(lactic acid) and/or poly(glycolic acid)) poly[bis(p-carboxyphenoxy) propane anhydride] (PCPP), poly[bis(p-carboxy)methane anhydride] (PCPM), polyanhydrides of oligomerized unsaturated aliphatic acids, polyanhydride polymers prepared from amino acids which are modified to include an additional carboxylic acid, aromatic polyanhydride compositions, and co-polymers of polyanhydrides with other substances, such as fatty acid terminated polyanhydrides, e.g., polyanhydrides polymerized from monomers of dimers and/or trimers of unsaturated fatty acids or unsaturated aliphatic acids. Polyanhydrides may be prepared in accordance with the methods set forth in U.S. Pat. No. 4,757,128, incorporated herein by reference. Polyorthoester polymers may be prepared, e.g., as set forth in U.S. Pat. No. 4,070,347, incorporated herein by reference. Polyphosphoesters may be prepared and used as set forth in U.S. Pat. Nos. 6,008,318, 6,153,212, 5,952,451, 6,051,576, 6,103,255, 5,176,907 and 5,194,581, each of which is incorporated herein by reference.

Proteinaceous polymers may also be used. Proteinaceous polymers and their soluble derivatives include gelation biodegradable synthetic polypeptides, elastin, alkylated collagen, alkylated elastin, and the like. Biodegradable synthetic polypeptides include poly-(N-hydroxyalkyl)-L-asparagine, poly-(N-hydroxyalkyl)-L-glutamine, copolymers of N-hydroxyalkyl-L-asparagine and N-hydroxyalkyl-L-glutamine with other amino acids. Suggested amino acids include L-alanine, L-lysine, L-phenylalanine, L-valine, L-tyrosine, and the like.

In additional embodiments, the controlled release material, which in effect acts as a carrier for the compound of the invention, alone or in combination with one or more biologically active agents as described herein, can further include a bioadhesive polymer such as pectins (polygalacturonic acid), mucopolysaccharides (hyaluronic acid, mucin) or non-toxic lectins or the polymer itself may be bioadhesive, e.g., polyanhydride or polysaccharides such as chitosan.

In embodiments where the biodegradable polymer comprises a gel, one such useful polymer is a thermally gelling polymer, e.g., polyethylene oxide, polypropylene oxide (PEO-PPO) block copolymer such as Pluronic™ F127 from BASF Wyandotte. In such cases, the local anesthetic formulation may be injected via syringe as a free-flowing liquid, which gels rapidly above 30° C. (e.g., when injected into a patient). The gel system then releases a steady dose of the compound of the invention, alone or in combination with one or more biologically active agents as described herein, at the site of administration.

### Solid Dosage Forms for Oral Use

Formulations for oral use include tablets comprising the compound of the invention with or without one or more of the biologically active agents described herein in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The compound of the invention and a biologically active agent, as defined herein, may be mixed together in a tablet, capsule, or other vehicle, or may be partitioned. In one example, the compound of the invention is contained on the inside of the tablet, and the biologically active agent is on the outside of the tablet, such that a substantial portion of the biologically active agent is released prior to the release of the compound of the invention.

Formulations for oral use may also be provided as chewable tablets, or as hard gelatin capsules wherein the compound of the invention is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion controlled release can be achieved by appropriate coating of a tablet, capsule, pellet, or granulate formulation of the compound of the invention, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compound and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the combination of the invention will depend on the nature of the compound, and can readily be determined by one skilled in the art. Typically, such dosage is normally about 0.001 mg to 2000 mg per day, desirably about 1 mg to 1000 mg per day, and more desirably about 5 mg to 500 mg per day. Dosages up to 200 mg per day may be necessary.

Administration of each drug in a combination therapy, as described herein, can, independently, be one to four times daily for one day to one year, and may even be for the life of the patient. Chronic, long-term administration will be indicated in many cases.

### Parenteral Formulations

Formulations suitable for parenteral administration (e.g., by injection), include aqueous or non-aqueous, isotonic, pyrogen-free, sterile liquids (e.g., solutions, suspensions), in which the compound of the invention is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound of the invention in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Topical Formulations

The composition of the invention, alone or in combination with one or more of the biologically active agents described herein, can also be adapted for topical use with a topical vehicle containing from between 0.0001% and 25% (w/w) or more of active ingredient(s).

In a preferred combination, the active ingredients are preferably each from between 0.0001% to 10% (w/w), more preferably from between 0.0005% to 4% (w/w) active agent. The cream can be applied one to four times daily, or as needed. Performing the methods described herein, the topical vehicle containing the composition of the invention, or a combination therapy containing a composition of the invention is preferably applied to the site of inflammation on the patient. For example, a cream may be applied to the hands of a patient suffering from arthritic fingers.

The compositions can be formulated using any dermatologically acceptable carrier. Exemplary carriers include a solid carrier, such as alumina, clay, microcrystalline cellulose, silica, or talc; and/or a liquid carrier, such as an alcohol, a glycol, or a water-alcohol/glycol blend. The therapeutic agents may also be administered in liposomal formulations that allow therapeutic agents to enter the skin. Such liposomal formulations are described in U.S. Pat. Nos. 5,169,637; 5,000,958; 5,049,388; 4,975,282; 5,194,266; 5,023,087; 5,688,525; 5,874,104; 5,409,704; 5,552,155; 5,356,633; 5,032,582; 4,994,213; 8,822,537, and PCT Publication No. WO 96/40061. Examples of other appropriate vehicles are described in U.S. Pat. Nos. 4,877,805, 8,822,537, and EP Publication No. 0586106A1. Suitable vehicles of the invention may also include mineral oil, petrolatum, polydecene, stearic acid, isopropyl myristate, polyoxyl 40 stearate, stearyl alcohol, or vegetable oil.

The composition can further include a skin penetrating enhancer, such as those described in “Percutaneous Penetration enhancers”, (eds. Smith E W and Maibach H I. CRC Press 1995). Exemplary skin penetrating enhancers include alkyl (N,N-disubstituted amino alkanoate) esters, such as dodecyl 2-(N,N dimethylamino) propionate (DDAIP), which is described in patents U.S. Pat. Nos. 6,083,996 and 6,118,020, which are both incorporated herein by reference; a water-dispersible acid polymer, such as a polyacrylic acid polymer, a carbomer (e.g., Carbopol™ or Carbopol 940P™, available from B. F. Goodrich Company (Akron, Ohio)), copolymers of polyacrylic acid (e.g., Pemulen™ from B. F. Goodrich Company or Polycarbophil™ from A. H. Robbins, Richmond, Va.; a polysaccharide gum, such as agar gum, alginate, carrageenan gum, ghatti gum, karaya gum, kadaya gum, rhamsan gum, xanthan gum, and galactomannan gum (e.g., guar gum, carob gum, and locust bean gum), as well as other gums known in the art (see for instance, Industrial Gums: Polysaccharides & Their Derivatives, Whistler R. L., BeMiller J. N. (eds.), 3rd Ed. Academic Press (1992) and Davidson, R. L., Handbook of Water-Soluble Gums & Resins, McGraw-Hill, Inc., N.Y. (1980)); or combinations thereof.

Other suitable polymeric skin penetrating enhancers are cellulose derivatives, such as ethyl cellulose, methyl cellulose, hydroxypropyl cellulose. Additionally, known transdermal penetrating enhancers can also be added, if desired. Illustrative are dimethyl sulfoxide (DMSO) and dimethyl acetamide (DMA), 2-pyrrolidone, N,N-diethyl-m-toluamide (DEET), 1-dodecylazacycloheptane-2-one (Azone™, a registered trademark of Nelson Research), N,N-dimethylformamide, N-methyl-2-pyrrolidone, calcium thioglycolate and other enhancers such as dioxolanes, cyclic ketones, and their derivatives and so on.

Also illustrative are a group of biodegradable absorption enhancers which are alkyl N,N-2-(disubstituted amino) alkanoates as described in U.S. Pat. Nos. 4,980,378 and 5,082,866, which are both incorporated herein by reference, including: tetradecyl (N,N-dimethylamino) acetate, dodecyl (N,N-dimethylamino) acetate, decyl (N,N-dimethylamino) acetate, octyl (N,N-dimethylamino) acetate, and dodecyl (N,N-diethylamino) acetate.

Particularly preferred skin penetrating enhancers include isopropyl myristate; isopropyl palmitate; dimethyl sulfoxide; decyl methyl sulfoxide; dimethylalanine amide of a medium chain fatty acid; dodecyl 2-(N,N-dimethylamino) propionate or salts thereof, such as its organic (e.g., hydrochloric, hydrobromic, sulfuric, phosphoric, and nitric acid addition salts) and inorganic salts (e.g., acetic, benzoic, salicylic, glycolic, succinic, nicotinic, tartaric, maleic, malic, pamoic, methanesulfonic, cyclohexanesulfamic, picric, and lactic acid addition salts), as described in U.S. Pat. No. 6,118,020; and alkyl 2-(N,N-disubstituted amino)-alkanoates, as described in U.S. Pat. Nos. 4,980,378 and 5,082,866.

The skin penetrating enhancer in this composition by weight would be in the range of 0.5% to 10% (w/w). The most preferred range would be between 1.0% and 5% (w/w). In another embodiment, the skin penetrating enhancer comprises between 0.5%-1%, 1%-2%, 2%-3%, 3%-4%, or 4%-5%, (w/w) of the composition.

The compositions can be provided in any useful form. For example, the compositions of the invention may be formulated as solutions, emulsions (including microemulsions), suspensions, creams, foams, lotions, gels, powders, or other typical solid, semi-solid, or liquid compositions (e.g., topical sprays) used for application to the skin or other tissues where the compositions may be used. Such compositions may contain other ingredients typically used in such products, such as colorants, fragrances, thickeners (e.g., xanthan gum, a fatty acid, a fatty acid salt or ester, a fatty alcohol, a modified cellulose, a modified mineral material, Krisgel 100™, or a synthetic polymer), antimicrobials, solvents, surfactants, detergents, gelling agents, antioxidants, fillers, dyestuffs, viscosity-controlling agents, preservatives, humectants, emollients (e.g., natural or synthetic oils, hydrocarbon oils, waxes, or silicones), hydration agents, chelating agents, demulcents, solubilizing excipients, adjuvants, dispersants, skin penetrating enhancers, plasticizing agents, preservatives, stabilizers, demulsifiers, wetting agents, sunscreens, emulsifiers, moisturizers, astringents, deodorants, and optionally including anesthetics, anti-itch actives, botanical extracts, conditioning agents, darkening or lightening agents, glitter, humectants, mica, minerals, polyphenols, silicones or derivatives thereof, sunblocks, vitamins, and phytomedicinals. The compositions can also include other like ingredients to provide additional benefits and improve the feel and/or appearance of the topical formulation. Specific classes of additives commonly use in these formulations include: isopropyl myristate, sorbic acid NF powder, polyethylene glycol, phosphatidylcholine (including mixtures of phosphatidylcholine, such as phospholipon G), Krisgel 100™ distilled water, sodium hydroxide, decyl methyl sulfoxide (as a skin penetrating enhancer), menthol crystals, lavender oil, butylated hydroxytoluene, ethyl diglycol reagent, and 95% percent (190 proof) ethanol.

### Formulations for Ophthalmic Administration

The compound of the invention can also be formulated with an ophthalmically acceptable carrier in sufficient concentration so as to deliver an effective amount of the compound to the optic nerve site of the eye. Preferably, the ophthalmic, therapeutic solutions contain the compound of the invention with or without biologically active compounds as defined herein in a concentration range of approximately 0.0001% to approximately 1% (weight by volume) and more preferably approximately 0.0005% to approximately 0.1% (weight by volume).

An ophthalmically acceptable carrier does not cause significant irritation to the eye and does not abrogate the pharmacological activity and properties of the charged sodium channel blockers.

Ophthalmically acceptable carriers are generally sterile, essentially free of foreign particles, and generally have a pH in the range of 5-8. Preferably, the pH is as close to the pH of tear fluid (7.4) as possible. Ophthalmically acceptable carriers are, for example, sterile isotonic solutions such as isotonic sodium chloride or boric acid solutions. Such carriers are typically aqueous solutions contain sodium chloride or boric acid. Also useful are phosphate buffered saline (PBS) solutions.

Various preservatives may be used in the ophthalmic preparation. Preferred preservatives include, but are not limited to, benzalkonium potassium, chlorobutanol, thimerosal, phenylmercuric acetate, and phenylmercuric nitrate. Likewise, various preferred vehicles may be used in such ophthalmic preparation. These vehicles include, but are not limited to, polyvinyl alcohol, povidone, hydroxypropyl methyl cellulose, poloxamers, carboxymethyl cellulose and hydroxyethyl cellulose.

Tonicity adjustors may be added as needed or convenient. They include, but are not limited to, salts, particularly sodium chloride, potassium chloride, etc., mannitol and glycerin, or any other suitable ophthalmically acceptable tonicity adjustor.

Various buffers and means for adjusting pH may be used so long as the resulting preparation is ophthalmically acceptable. Accordingly, buffers include but are not limited to, acetate buffers, citrate buffers, phosphate buffers, and borate buffers. Acids or bases may be used to adjust the pH of these formulations as needed. Ophthalmically acceptable antioxidants can also be include. Antioxidants include but are not limited to sodium metabisulfite, sodium thiosulfate, acetylcysteine, butylated hydroxyanisole, and butylated hydroxytoluene.

### Formulations for Nasal and Inhalation Administration

The pharmaceutical compositions of the invention can be formulated for nasal or intranasal administration. Formulations suitable for nasal administration, when the carrier is a solid, include a coarse powder having a particle size, for example, in the range of approximately 20 to 500 microns which is administered by rapid inhalation through the nasal passage. When the carrier is a liquid, for example, a nasal spray or as nasal drops, one or more of the formulations can be admixed in an aqueous or oily solution, and inhaled or sprayed into the nasal passage.

For administration by inhalation, the active ingredient can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, for example, gelatin for use in an inhaler or insufflator can be formulated containing a powder mix of the compound of the invention and a suitable powder base such as lactose or starch.

### Indications

The methods, compositions, and kits of the invention can be used to treat pain or itch associated with any of a number of conditions, including back and neck pain, cancer pain, gynecological and labor pain, fibromyalgia, arthritis and other rheumatological pains, orthopedic pains, post herpetic neuralgia and other neuropathic pains, sickle cell crises, interstitial cystitis, urethritis and other urological pains, dental pain, headaches, postoperative pain, and procedural pain (i.e., pain associated with injections, draining an abcess, surgery, dental procedures, opthalmic procedures, ophthalmic irritation, conjuctivitis (e.g., allergic conjunctivitis), eye redness, dry eye, arthroscopies and use of other medical instrumentation, cosmetic surgical procedures, dermatological procedures, setting fractures, biopsies, and the like).

Since a subclass of nociceptors mediate itch sensation the methods, compositions, and kits of the invention can also be used to treat itch in patients with conditions like dermatitis, infections, parasites, insect bites, pregnancy, metabolic disorders, liver or renal failure, drug reactions, allergic reactions, eczema, and cancer.

The methods, compositions, and kits of the invention can also be used for the treatment of pyrexia (fever), hyperpyrexia, malignant hyperthermia, or a condition characterized by elevated body temperature.

The methods, compositions, and kits of the invention can also be used to treat neurogenic inflammation and neurogenic inflammatory disorders. Inflammation is a complex set of responses to harmful stimuli that results in localized redness, swelling, and pain. Inflammation can be innate or adaptive, the latter driven by antigens and is mediated by immune cells (immune-mediated inflammation). Neurogenic inflammation results from the efferent functions of pain-sensing neurons (nociceptors), wherein neuropeptides and other chemicals that are pro-inflammatory mediators are released from the peripheral terminals of the nociceptors when they are activated. This release process is mediated by calcium influx and exocytosis of peptide containing vesicles, and the pro-inflammatory neuropeptides include substance P, neurokinin A and B (collectively known as tachykinins), calcitonin gene-related peptide (CGRP), and vasoactive intestinal polypeptide (VIP).

The release of peripheral terminal chemicals stimulate a variety of inflammatory responses. First, the release of substance P can result in an increase in capillary permeability such that plasma proteins leak from the intravascular compartment into the extracellular space (plasma extravasation), causing edema. This can be detected as a wheal (a firm, elevated swelling of the skin) which is one component of a triad of inflammatory responses—wheal, red spot, and flare—known as the Lewis triple response. Second, the release of CGRP causes vasodilation, leading to increased blood flow. This can be detected as a flare, which is another component of the Lewis triple response.

Substance P also has a pro-inflammatory action on immune cells (e.g. macrophages, T-cells, mast cells, and dendritic cells) via their neurokinin-1 (NK1) receptor. This effect has been documented in allergic rhinitis, gastitis, and colitis, and represents an interface between the neurogenic and immune-mediated components of inflammation. Substance P released from one nociceptor may also act on NK1 receptors on neighboring nociceptors to sensitize or activate them, causing a spread of activation and afferent/efferent function. These efferent functions of nociceptors can be triggered by: 1) Direct activation of a nociceptor terminal by a peripheral adequate stimulus applied to the terminal (e.g. a pinch); 2) Indirect antidromic activation of a non-stimulated nociceptor terminal by the axon reflex, wherein action potential input from one terminal of a nociceptor, upon reaching a converging axonal branch point in the periphery, results in an action potential traveling from the branch point down to the peripheral terminal of a non-stimulated terminal; and 3) Activation as a result of activity in nociceptor central terminals in the CNS traveling to the periphery (e.g., primary afferent depolarization of central terminals produced by GABA can be sufficient to initiate action potentials traveling the “wrong way”).

Genomic analysis of lung resident ILC2 cells has revealed expression of receptors for several neuropeptides released by sensory neurons, including SP, CGRP and VIP, providing an opportunity for nociceptors to directly communicate with these cells. In particular, VIP is found to be expressed in NaV1.8+ nodose ganglion neurons, including lung afferents in OVA-exposed mice. Cultured nodose ganglion neurons stimulated with capsaicin or IL5 also released VIP while BALF from OVA-exposed mice contained elevated VIP compared to vehicle-challenged mice (Talbot et al., Neuron 2015, in press). These data indicate that VIP is released in the inflamed lung and can be blocked by silencing neurons with charged sodium channel blockers of the present invention. In addition, when CD4+ T cells cultured under TH2 skewing conditions were exposed to recombinant mouse VIP, the transcript levels of IL-13 and IL-5 increased, suggesting that VIP contributes to the competence of TH2 cells to transcribe these type II regulatory cytokines.

Immune mediator release from immune cells can also activate nociceptors. Mast cells are found close to primary nociceptive neurons and contribute to nociceptor sensitization in a number of contexts. Injection of the secretagogue compound 48/80 promotes degranulation of mast cells in the dura and leads to excitation of meningeal nociceptors. Mast cell degranulation also contributes to the rapid onset of nerve growth factor-induced thermal hyperalgesia. Macrophages contribute to nociceptor sensitization by releasing several soluble mediators. Expression of the chemokine macrophage inflammatory protein-1a (MIP-1a) and its receptors CCR1 and CCR5 is increased in macrophages and Schwann cells after partial ligation of the sciatic nerve and contributes to the development of neuropathic pain. Lymphocytes contribute to the sensitization of peripheral nociceptors. T cells infiltrate the sciatic nerve and dorsal root ganglion (DRG) after nerve injury. Hyperalgesia and allodynia induced by nerve injury are markedly attenuated or abrogated in rodents lacking T cells and the immunosuppressant rapamycin attenuates neuropathic pain in rats, partly owing to an effect on T cells. Among the subsets of T cells, type 1 and 2 helper T cells (TH1 and TH2 cells) have been shown to have different roles in neuropathic pain. TH1 cells facilitate neuropathic pain behavior by releasing proinflammatory cytokines (IL-2 and interferon-γ (IFNγ)), whereas TH2 cells inhibit it by releasing anti-inflammatory cytokines (IL-4, IL-10 and IL-13). The complement system also has a role in inflammatory hyperalgesia and neuropathic pain. C5a, an anaphylatoxin, is an important effector of the complement cascade and upon binding to C5aR1 receptors on neutrophils it becomes a potent neutrophil attractant (Ren & Dubner, Nat. Med. 16:1267-1276 (2010)).

Bacterial infections have been shown to directly activate nociceptors, and that the immune response mediated through TLR2, MyD88, T cells, B cells, and neutrophils and monocytes is not necessary for Staphylococcus aureus-induced pain in mice (Chiu et al., Nature 501:52-57 (2013)). Mechanical and thermal hyperalgesia in mice is correlated with live bacterial load rather than tissue swelling or immune activation. Bacteria induce calcium flux and action potentials in nociceptor neurons, in part via bacterial N-formylated peptides and the pore-forming toxin α-haemolysin, through distinct mechanisms. Specific ablation of Nav1.8-lineage neurons, which include nociceptors, abrogated pain during bacterial infection, but concurrently increased local immune infiltration and lymphadenopathy of the draining lymph node. Thus, bacterial pathogens produce pain by directly activating sensory neurons that modulate inflammation, an unsuspected role for the nervous system in host-pathogen interactions. Data from Talbot et al., Neuron 2015, in press have also suggested that nociceptors are activated during exposure to allergens in sensitized animals.

In certain disorders, neurogenic inflammation contributes to the peripheral inflammation elicited by tissue injury, autoimmune disease, infection, and exposure to irritants in soft tissue, skin, the respiratory system, joints, the urogenital and GI tract, the liver, and the brain. Neurogenic inflammatory disorders include asthma, rhinitis, conjunctivitis, arthritis, colitis, contact dermatitis, diabetes, eczema, cystitis, gastritis, migraine headache, psoriasis, rhinitis, rosacea, and sunburn. pancreatitis, chronic cough, chronic rhinosinusistis, traumatic brain injury, polymicrobial sepsis, tendinopathies chronic urticaria, rheumatic disease, acute lung injury, exposure to irritants, inhalation of irritants, pollutants, or chemical warfare agents, as described herein.

### Assessment of Pain, Itch, and Neurogenic Inflammation

In order to measure the efficacy of any of the methods, compositions, or kits of the invention, a measurement index may be used. Indices that are useful in the methods, compositions, and kits of the invention for the measurement of pain associated with musculoskeletal, immunoinflammatory and neuropathic disorders include a visual analog scale (VAS), a Likert scale, categorical pain scales, descriptors, the Lequesne index, the WOMAC index, and the AUSCAN index, each of which is well known in the art. Such indices may be used to measure pain, itch, function, stiffness, or other variables. A visual analog scale (VAS) provides a measure of a one-dimensional quantity. A VAS generally utilizes a representation of distance, such as a picture of a line with hash marks drawn at regular distance intervals, e.g., ten 1-cm intervals. For example, a patient can be asked to rank a sensation of pain or itch by choosing the spot on the line that best corresponds to the sensation of pain or itch, where one end of the line corresponds to “no pain” (score of 0 cm) or “no itch” and the other end of the line corresponds to “unbearable pain” or “unbearable itch” (score of 10 cm). This procedure provides a simple and rapid approach to obtaining quantitative information about how the patient is experiencing pain or itch. VAS scales and their use are described, e.g., in U.S. Pat. Nos. 6,709,406 and 6,432,937.

A Likert scale similarly provides a measure of a one-dimensional quantity. Generally, a Likert scale has discrete integer values ranging from a low value (e.g., 0, meaning no pain) to a high value (e.g., 7, meaning extreme pain). A patient experiencing pain is asked to choose a number between the low value and the high value to represent the degree of pain experienced. Likert scales and their use are described, e.g., in U.S. Pat. Nos. 6,623,040 and 6,766,319.

The Lequesne index and the Western Ontario and McMaster Universities (WOMAC) osteoarthritis index assess pain, function, and stiffness in the knee and hip of OA patients using self-administered questionnaires. Both knee and hip are encompassed by the WOMAC, whereas there is one Lequesne questionnaire for the knee and a separate one for the hip. These questionnaires are useful because they contain more information content in comparison with VAS or Likert. Both the WOMAC index and the Lequesne index questionnaires have been extensively validated in OA, including in surgical settings (e.g., knee and hip arthroplasty). Their metric characteristics do not differ significantly.

The AUSCAN (Australian-Canadian hand arthritis) index employs a valid, reliable, and responsive patient self-reported questionnaire. In one instance, this questionnaire contains 15 questions within three dimensions (Pain, 5 questions; Stiffness, 1 question; and Physical function, 9 questions). An AUSCAN index may utilize, e.g., a Likert or a VAS scale.

Indices that are useful in the methods, compositions, and kits of the invention for the measurement of pain include the Pain Descriptor Scale (PDS), the Visual Analog Scale (VAS), the Verbal Descriptor Scales (VDS), the Numeric Pain Intensity Scale (NPIS), the Neuropathic Pain Scale (NPS), the Neuropathic Pain Symptom Inventory (NPSI), the Present Pain Inventory (PPI), the Geriatric Pain Measure (GPM), the McGill Pain Questionnaire (MPQ), mean pain intensity (Descriptor Differential Scale), numeric pain scale (NPS) global evaluation score (GES) the Short-Form McGill Pain Questionnaire, the Minnesota Multiphasic Personality Inventory, the Pain Profile and Multidimensional Pain Inventory, the Child Heath Questionnaire, and the Child Assessment Questionnaire. Itch can be measured by subjective measures (VAS, Lickert, descriptors). Another approach is to measure scratch which is an objective correlate of itch using a vibration transducer or movement-sensitive meters.

The following examples are intended to illustrate the invention and are not intended to limit it.

### EXAMPLES

### Example 1—Synthesis of 1-(1-(2, 6-dimethylphenylamino)-1-oxobutan-2-yl)-1-propylpiperidinium chloride (Compound 1)

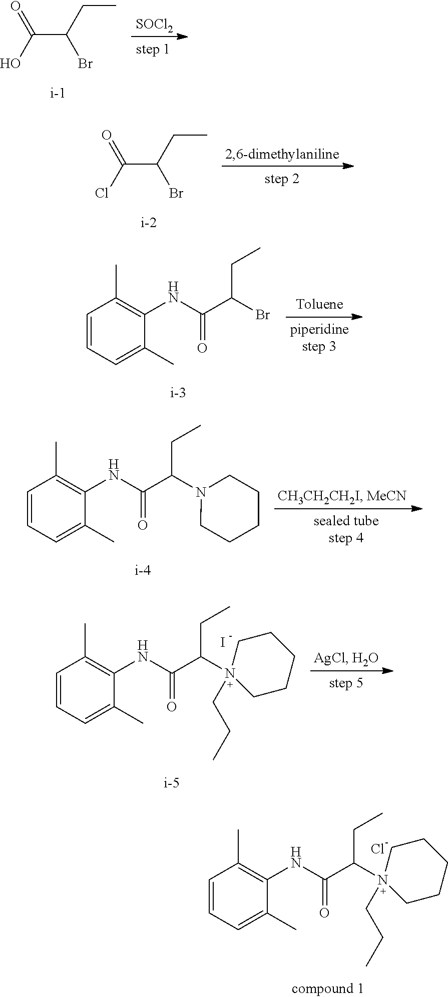

### Step 1: Preparation of Intermediate i-1

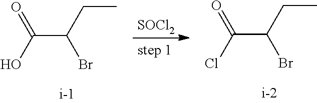

The solution of i-1 (200.0 g, 1200 mmol, 1.0 eq) in 1200 mL SOCl₂ was refluxed at 80° C. for 2 hours. After competition, the reaction mixture was directly concentrated in vacuum to give a residue without further purification (193 g, 87% yield).

### Step 2: Preparation of Intermediate i-3

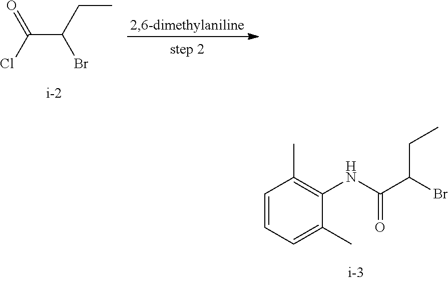

To the solution of i-2, 6-dimethylaniline (106 g, 870 mmol, 1.0 equiv), and pyridine (103 g, 1300 mmol, 1.5 equiv) in DCM (3000 mL) in an ice bath, i-2 (193.0 g, 1050 mmol, 1.2 equiv) in DCM (200 mL) was added slowly. Then the reaction mixture was warmed to room temperature for 2 hours. After competition, the reaction mixture was adjusted to pH 5-6 with 2 N HCl and extracted with H₂O (1000 mL). The combined organic phases was washed with hexanes (600 g), dried over Na₂SO₄, filtered, and concentrated in vacuum to give a residue. The residue was purified by column chromatography to give the desired product (194.2 g, 83% yield) as a yellow solid. HPLC purity: 97% at 220 nm. Mass: m/z=270 (M+1, ESI+).

### Step 3: Preparation of Intermediate i-4

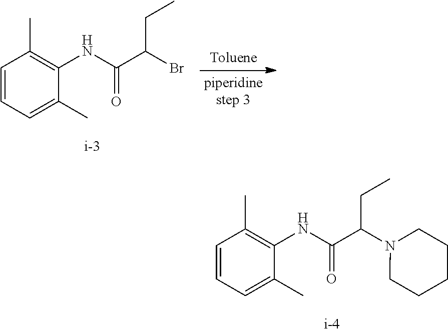 [SMILES: CCC(Br)C(=O)Nc1c(C)cccc1C ; CCC(C(=O)Nc1c(C)cccc1C)N1CCCCC1]

To the solution of i-3 (50.0 g, 185.8 mmol, 1.0 equiv) in toluene (929 mL, c=0.2 M) was added piperidine (33.2 g, 390.3 mmol, 2.1 equiv). The reaction mixture was refluxed at 110° C. overnight. After competition, the reaction mixture was filtered and concentrated in vacuum to give a residue. The residue was washed with n-hexane to give the desired product (35.0 g, 68% yield) as a white solid. HPLC purity: 97.4% at 220 nm. Mass: m/z=275 (M+1, ESI+).

### Step 4: Preparation of Intermediate i-5

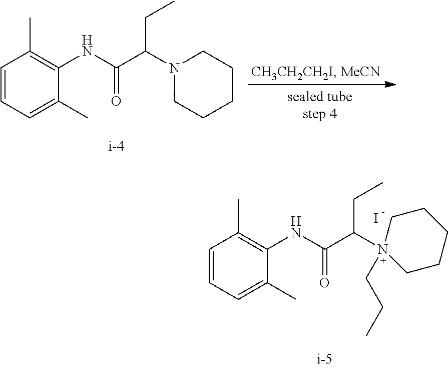

Intermediate i-4 (35 g, 127.6 mmol, 1.0 equiv) was dissolved in MeCN (127.6 mL). To this was added 1-iodopropane (108.4 g). The mixture was heated at 80° C. in a sealed tube for 20 hours. The reaction mixture was then concentrated in vacuum to give the desired product (17.2 g, 29% yield) as a white solid. HPLC purity: 96.8% at 220 nm. Mass: m/z=318 (M+1, ESI+).

### Step 5: Preparation of Compound 1

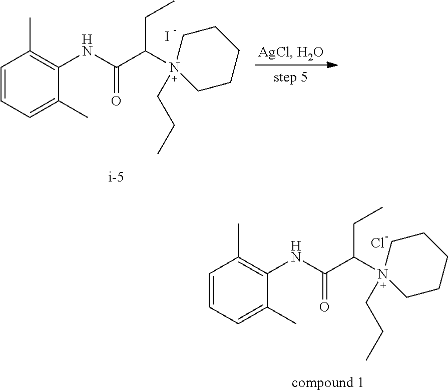

To a solution of i-4 (17 g, 38.3 mmol, 1.0 equiv) in H₂O (191 mL) was added AgCl (10.9 g). The mixture was heated at 65° C. for 2 hours, then filtered and lyophilized to give the desired product (13 g, 96% yield) as a white solid. ¹HNMR (400 MHz, DMSO) δ 10.73 (s, 1H), 7.14-7.10 (m, 3H), 4.53-4.50 (d, J=10.0 Hz, 1H), 3.79-3.68 (m, 3H), 3.44-3.33 (m, 3H), 2.22-2.14 (m, 8H), 2.00-1.78 (m, 8H), 1.12-1.09 (t, J=7.4 Hz, 3H), 0.99-0.95 (t, J=7.2 Hz, 3H). HPLC purity: 99% at 220 nm. Mass: m/z=318.75 (M+1, ESI+).

### Example 2—Synthesis of 1-(1-((2, 6-dimethylphenyl) amino)-1-oxopentan-2-yl)-1-ethylpiperidin-1-ium (Compound 2)

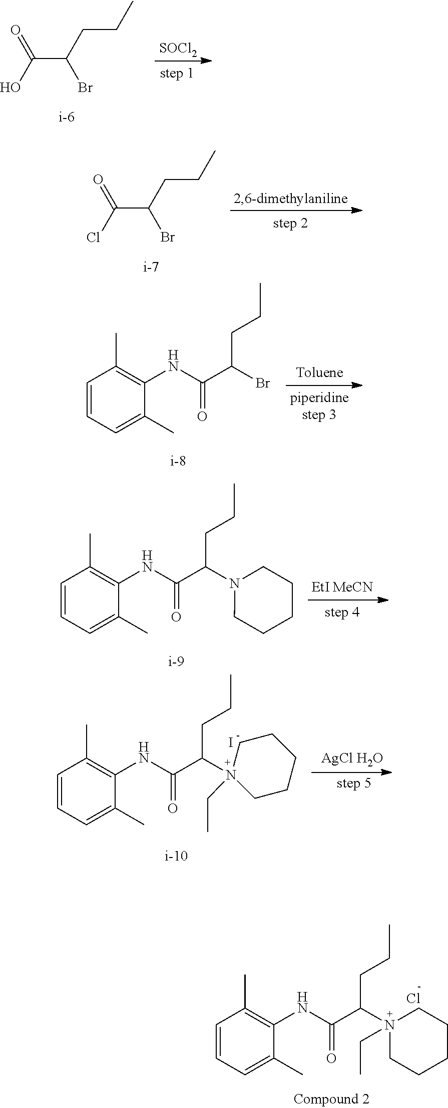

### Step 1: Preparation of Intermediate i-7

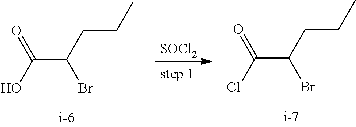

The solution of i-6 (20.0 g, 111.13 mmol, 1.0 equiv) in SOCl₂ (13.9 g) was refluxed at 80° C. for 2 hours. After competition, the reaction mixture was directly concentrated in vacuum to give a residue without further purification (22 g, quantitative yield).

### Step 2: Preparation of Intermediate i-8

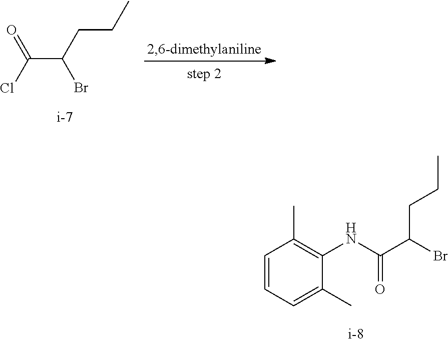

To the solution of 2,6-dimethylaniline (9.3 g, 77.12 mmol, 1.0 euivq) in DCM (230 mL) and pyridine (18.3 g, 231.36 mmol, 3.0 equiv) in an ice bath, i-7 (22 g, 111.13 mmol, 1.5 eq) in DCM (100 mL) was slowly added. Then the reaction mixture was warmed to room temperature for 2 hours. After completion, the reaction mixture was adjusted pH 5-6 with 2 N HCl and then extracted with ethyl acetate (200 mL×2). The combined organic phases was washed with brine (150 mL), dried over Na₂SO₄, filtered, and concentrated in vacuum to give a residue. The residue was purified by column chromatography to give the desired product (17.4 g, 80% yield) as yellow solid.

### Step 3: Preparation of Intermediate i-9

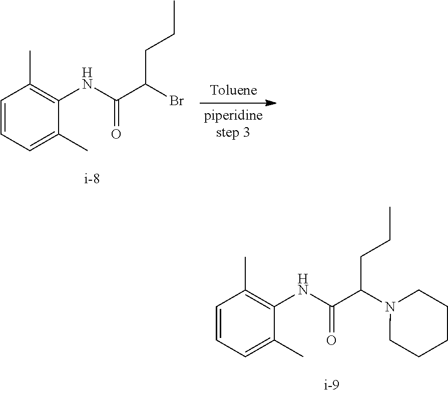 [SMILES: CCCC(Br)C(=O)Nc1c(C)cccc1C ; CCCC(C(=O)Nc1c(C)cccc1C)N1CCCCC1]

To a solution of i-8 (24.7 g, 87.26 mmol, 1.0 equiv) in toluene (436 mL, c=0.2 M) was added piperidine (15.59 g, 183.24 mmol, 2.1 equiv). The reaction mixture was refluxed at 110° C. for 3 hours. After competition, the reaction mixture was directly concentrated in vacuum to give a residue. The residue was purified by column chromatography to give the desired product (18.0 g, 72% yield) as a white solid.

### Step 4: Preparation of Intermediate i-10

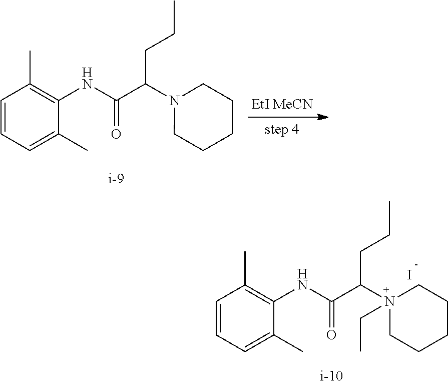

To a solution of i-9 (11.82 g, 41.03 mmol, 1.0 equiv) in MeCN (103 mL, c=0.4 M) was added ethyl iodide (19.2 g, 123.07 mmol, 3.0 equiv). The reaction mixture was stirred at 85° C. overnight. The reaction mixture was then directly concentrated in vacuum to give a residue. The residue was washed with ethyl acetate to give the desired product (9.99 g, 54.8% yield). HPLC purity: 98.2% at 220 nm.

### Step 5: Preparation of Compound 2

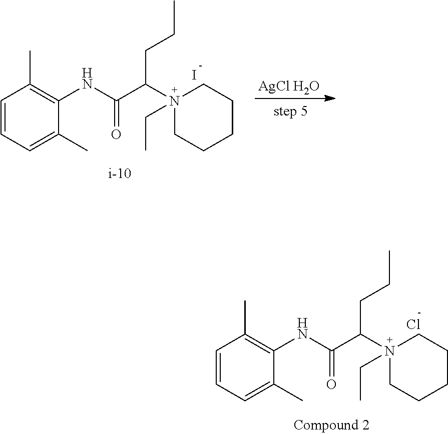

To the solution of i-10 (9.99 g, 22.49 mmol, 1.0 equiv) in H₂O (75 mL, c=0.3 M) was added AgCl (6.47 g, 2.0 equiv). The reaction mixture was heated at 60° C. overnight. After competition, the reaction mixture was filtered. The filtrate was lyophilized to give the desired product. (7.23 g, 91% yield) as a white solid. ¹H NMR (400 MHz, DMSO) δ 10.60 (s, 1H), 7.18-7.10 (m, 3H), 4.50-4.40 (t, 1H), 3.95-3.82 (m, 1H), 3.80-3.70 (m, 2H), 3.60 (m, 1H), 3.45-3.35 (m, 2H), 2.25-2.15 (m, 6H), 2.05 (m, 2H), 1.95-1.8 (m, 5H), 1.7 (m, 1H), 1.5 (m, 3H) 1.35 (t, 3H), 1.05 (t, 3H). HPLC purity: 99% at 220 nm. Mass: m/z=317.95 (M+1, ESI+).

### Example 3—Block of hNaᵥ1.7 Channels Expressed in HEK Cells by Compound 1

This example demonstrates that compound 1 has a greater efficacy than QX-314 for inhibiting Nav1.7 sodium channels when applied inside cells.

Procedure:

Whole-cell recordings were made of currents carried by voltage-activated sodium channels in HEK293 cells stably expressing human Nav1.7 channels. Recordings were made using patch pipettes with resistances of 2-3.5 MO when filled with internal solution, consisting of 61 mM CsF, 61 mM CsCl, 9 mM NaCl, 1.8 mM MgCl₂, 9 mM EGTA, 14 mM creatine phosphate (tris salt), 4 mM MgATP, and 0.3 mM GTP (tris salt), 9 mM HEPES, pH adjusted to 7.2 with CsOH. The shank of the electrode was wrapped with Parafilm in order to reduce capacitance and allow optimal series resistance compensation without oscillation. Seals were obtained and the whole-cell configuration established with cells in Tyrode's solution (155 mM NaCl, 3.5 mM KCl, 1.5 mM CaCl₂), 1 mM MgCl₂, 10 mM HEPES, 10 mM glucose, pH adjusted to 7.4 with NaOH) with 10 mM TEACl. To ensure complete dialysis with pipette solution containing the test compounds, recordings began after 5 to 10 minutes after establishment of the whole-cell configuration.

Currents were recorded at room temperature (21-23° C.) with an Axopatch 200 amplifier or Multiclamp 200B amplifier and filtered at 5-10 kHz with a low-pass Bessel filter. The amplifier was tuned for partial compensation of series resistance (typically 70-80% of a total series resistance of 4-10 MO). Currents were digitized using a Digidata 1322A data acquisition interface controlled by pClamp9 or pClamp10 software (Axon Instruments) and analyzed using programs written in Igor Pro 4.0 (Wavemetrics, Lake Oswego, Oreg.). Currents were corrected for linear capacitative and leak currents determined using 5 mV hyperpolarizations delivered from the resting potential (usually −80 or −100 mV) and then appropriately scaled and subtracted.

Sodium currents were evoked by 30-msec depolarizations from −100 mV to −20 mV. To assay use-dependent block, pulses were delivered at series of increasing rates: 0.05 Hz for 1-min, 0.33 Hz for 1-min, 1 Hz for 1-min, 3 Hz for 1-min, 5 Hz for 30 seconds, 10 Hz for 30 seconds, with 1 minute rest between each series of pulses. After the series of pulses to induce use-dependent block, the time course of recovery from use-dependent block was followed using pulses delivered at 0.1 Hz (2-min) and 0.05 Hz (1-min).

Results:

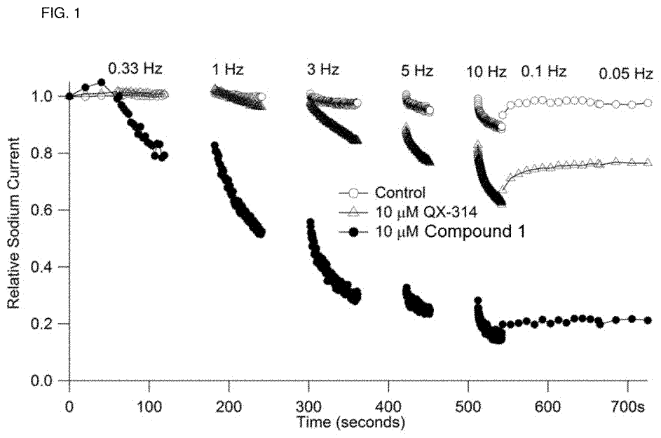

As shown in FIG. 1 both QX-314 and compound 1 at 10 micromolar show strong use-dependent accumulation of block at stimulation frequencies from 1 to 10 Hz, with only partial recovery during 1-minute rest intervals. QX-314 at 10 micromolar produced use-dependent inhibition of sodium current to about 62% of the initial value, followed by partial recovery to about 77% of the initial value after 3 minutes of slow stimulation. Compound 1 at 10 micromolar produced more profound use-dependent block, to about 15% of the initial sodium current, and recovered to about 22% of the initial value during 3 minutes of slow stimulation. Thus, compared with QX-314, compound 1 shows more accumulation of use-dependent block of Nav1.7 sodium channels.

### Example 4—Block of hNaᵥ1.7 Channels Expressed in HEK Cells by Compound 2

This example demonstrates that compound 2 has a greater efficacy than QX-314 for inhibiting Nav1.7 sodium channels when applied inside cells.

Procedure:

Whole-cell recordings were made of currents carried by voltage-activated sodium channels in HEK293 cells stably expressing human Nav1.7 channels. Recordings were made using patch pipettes with resistances of 2-3.5 MO when filled with internal solution, consisting of 61 mM CsF, 61 mM CsCl, 9 mM NaCl, 1.8 mM MgCl₂, 9 mM EGTA, 14 mM creatine phosphate (tris salt), 4 mM MgATP, and 0.3 mM GTP (tris salt), 9 mM HEPES, pH adjusted to 7.2 with CsOH. The shank of the electrode was wrapped with Parafilm in order to reduce capacitance and allow optimal series resistance compensation without oscillation. Seals were obtained and the whole-cell configuration established with cells in Tyrode's solution (155 mM NaCl, 3.5 mM KCl, 1.5 mM CaCl₂), 1 mM MgCl₂, 10 mM HEPES, 10 mM glucose, pH adjusted to 7.4 with NaOH) with 10 mM TEACl. To ensure complete dialysis with pipette solution containing the test compounds, recordings began after 5 to 10 minutes after establishment of the whole-cell configuration.

Currents were recorded at room temperature (21-23° C.) with an Axopatch 200 amplifier or Multiclamp 200B amplifier and filtered at 5-10 kHz with a low-pass Bessel filter. The amplifier was tuned for partial compensation of series resistance (typically 70-80% of a total series resistance of 4-10 MO). Currents were digitized using a Digidata 1322A data acquisition interface controlled by pClamp9 or pClamp10 software (Axon Instruments) and analyzed using programs written in Igor Pro 4.0 (Wavemetrics, Lake Oswego, Oreg.). Currents were corrected for linear capacitative and leak currents determined using 5 mV hyperpolarizations delivered from the resting potential (usually −80 or −100 mV) and then appropriately scaled and subtracted.

Sodium currents were evoked by 30-msec depolarizations from −100 mV to −20 mV. To assay use-dependent block, pulses were delivered at series of increasing rates: 0.05 Hz for 1-min, 0.33 Hz for 1-min, 1 Hz for 1-min, 3 Hz for 1-min, 5 Hz for 30 seconds, 10 Hz for 30 seconds, with 1 minute rest between each series of pulses. After the series of pulses to induce use-dependent block, the time course of recovery from use-dependent block was followed using pulses delivered at 0.1 Hz (2-min) and 0.05 Hz (1-min).

Results:

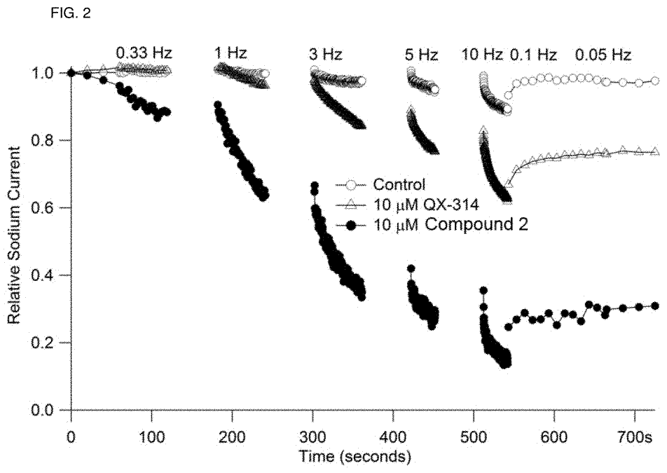

With cells dialyzed with control internal solution (open circles, FIG. 2), there was a small reduction of sodium current during the stimulation at 5 Hz or 10 Hz, representing accumulating inactivation of the channels, but the sodium current then recovered quickly and completely when the higher-frequency stimulation was stopped. Both QX-314 and compound 2 at 10 micromolar both show strong use-dependent accumulation of block at stimulation frequencies from 1 to 10 Hz, with only partial recovery during 1-minute rest intervals. QX-314 at 10 micromolar produced use-dependent inhibition of sodium current to about 62% of the initial value, followed by partial recovery to about 77% of the initial value after 3 minutes of slow stimulation. Compound 2 at 10 micromolar produced more profound use-dependent block, to about 14% of the initial sodium current, and recovered to about 31% of the initial value during 3 minutes of slow stimulation. Thus, compared with QX-314, compound 2 shows more accumulation of use-dependent block of Nav1.7 sodium channels.

### Example 5—Sodium Channel Block by Compound 1 Applied Extracellularly in the Absence of Large Pore Channels

This example shows that compound 1 at concentrations up to 1 mM has no blocking effect on Naᵥ1.7 and Naᵥ1.5 sodium channels when applied extracellularly in cells that do not have large pore channels allowing entry of the compounds (Naᵥ1.7 and Naᵥ1.5 expressed in HEK cells).

Procedure:

Charged sodium channel blockers were tested for extracellular block of voltage-gated sodium channels in a thallium flux assay. Briefly, human embryonic kidney (HEK293) cells stably expressing either the Naᵥ1.5 or Naᵥ1.7 sodium channels were plated in 384 well plates. After overnight culture at 37° C., the cell culture medium was replaced the thallium-sensitive FluxOR dye (FluxOR assay kit, Thermo Fisher). Following a 60 minute incubation at room temperature, the dye-loading solution was replaced with assay buffer and the plates were loaded into a FDSS 7000EX multi-well plate reader (Hamamatsu). Dye fluorescence was measured using Fluo-4 filters (excitation 470 nm, emission 540 nm) at 1 Hz. Baseline fluorescence was measured for 30 s, followed by the addition of 15 μL/well of charged sodium channel blocker compounds. 10 minutes after compound addition, the cells were stimulated with a Tl⁺ stimulus buffer and fluorescence measured. Tetraethylammonium (TEA) was used to block Kv channels and veratridine to block Naᵥ channel inactivation. The fluorescence data was normalized with respect to no veratridine stimulus buffer control in order to isolate and measure the Naᵥ channel component of the thallium flux.

Results:

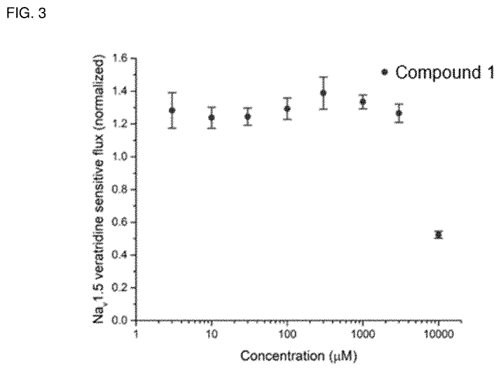

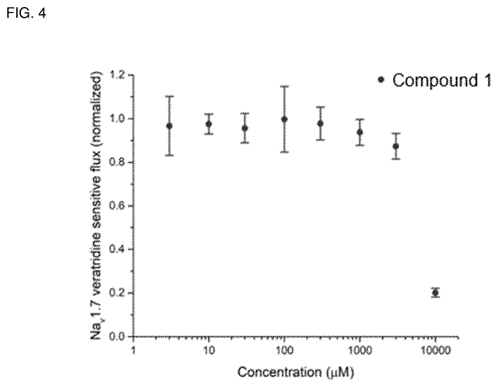

The Naᵥ1.7 channel is mainly expressed in somatosensory neurons, while the Naᵥ1.5 channel is primarily expressed in cardiomyocytes, and its extracellular block could be predictive of potential cardiotoxicity. Compound 1 was evaluated for extracellular block of Naᵥ1.5 and Naᵥ1.7 sodium channels in HEK cells via a thallium flux assay. Extracellular block of voltage-gated sodium currents (as monitored by thallium flux) by compound 1 only occurs in the high mM range. As shown in FIG. 3 and FIG. 4, compound 1 did not exhibit significant extracellular block of Naᵥ1.5 (see FIG. 3) or Naᵥ1.7 (see FIG. 4) at doses of up to 1 mM and did not display any Naᵥ subtype selectivity. These results show that compound 1 has no blocking effect on sodium channels up to at least 1 mM when applied extracellularly in cells that do not have large pore channels that would allow entry into the cells.

### Example 6—In vitro Electrophysiology of Sodium Channel Block in DRG Neurons

This example demonstrates that compound 1 applied extracellularly can permeate into sensory neurons through activated large pore channels to produce an intracellular sodium channel block.

Procedure:

Charged sodium channel blockers were tested for intracellular block of voltage-gated sodium currents after entry through activated TRPV1 channels in dissociated mouse dorsal root ganglia (DRG) neurons in vitro. DRG neurons from 8-week-old C57/B16 mice were removed and placed in DMEM containing 1% penicillin-streptomycin (Sigma), then treated for 90 minutes with 5 mg/mL collagenase, 1 mg/mL Dispase II (Roche). Cells were triturated in the presence of DNase I inhibitor (50 U), centrifuged through 10% BSA (Sigma), resuspended in 1 mL of Neurobasal medium (Sigma)+1X B27 supplement (Thermo Fisher) and 1% penicillin-streptomycin, and plated onto 35-mm tissue culture dishes (Becton Dickinson) coated with 20 μg/mL laminin, at 8,000-9,000 cells per dish. Cultures were incubated at 37° C. under 5% CO₂. Recordings were made at room temperature within 24 hours of plating. The DRG neurons were treated with 300 nM capsaicin alone or 300 nM capsaicin+100 μM charged sodium channel blocker in external solution for 30 minutes, after which the blocker was washed off with fresh external solution. Whole-cell voltage-clamp recordings of sodium currents were performed in small diameter DRG neurons and then the expression of TRPV1 measured by perfusion of 1 μM capsaicin.

Whole-cell voltage-clamp or current-clamp recordings were made with an Axopatch 200A amplifier (Molecular Devices) and patch pipettes with resistances of 2-4 MO. Cell capacitance was compensated for with the use of the amplifier circuitry, and linear leakage currents were subtracted with a P/4 procedure. Series resistance (less than 10 MO) was compensated for by about 80%. Voltage-clamp recordings used solutions designed to isolate sodium currents by blocking potassium and calcium currents and with a decreased external sodium concentration, to improve voltage clamp. Pipette solution was (in mM) 140 CsF, 10 NaCl, 2 MgCl₂, 0.1 CaCl₂, 1.1 EGTA, 10 HEPES, pH 7.2. The external solution was (in mM) 105 Choline-Cl, 35 NaCl, 20 TEA-Cl, 3 KCl, 1 MgCl₂, 1 CaCl₂, 0.1 CdCl₂, 10 glucose and 10 HEPES, pH 7.3. No correction was made for the liquid junction potential. Command protocols were generated and data were digitized with a Digidata 1200 A/D interface with pCLAMP 10.2 software (Molecular Devices). Voltage clamp current records were low-pass filtered at 2 kHz. Drugs were applied with a multibarrel drug delivery system placed 200-250 mm from the neuron. Solution exchange was complete in less than 1 s.

Results:

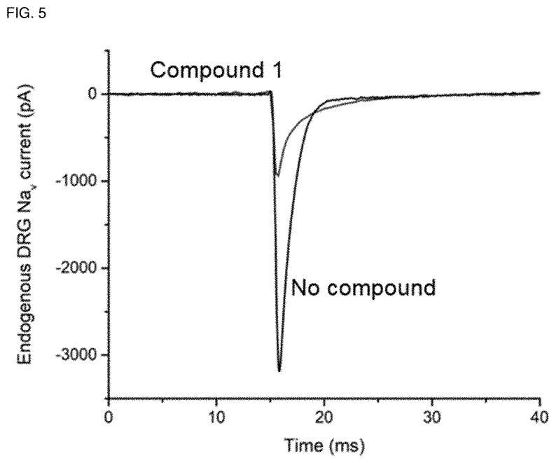

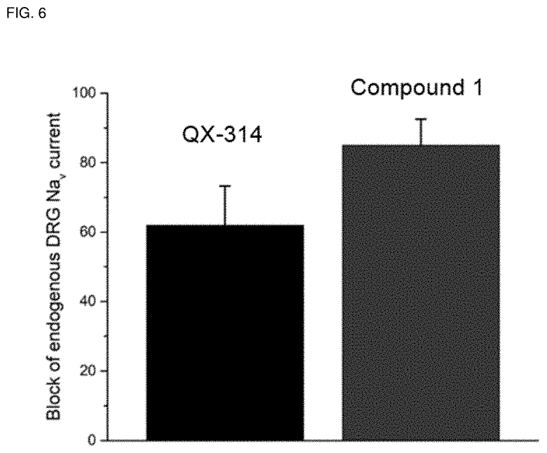

Compound 1 was tested to see if it could permeate into dorsal root ganglion (DRG) neurons through activated TRPV1 channels and thereby block endogenous Naᵥ currents in the neurons, comprised of Naᵥ1.7, Naᵥ1.8 and other sodium channels. Compound 1 was applied extracellularly together with capsaicin. Capsaicin is an agonist of large-pore TRPV1 channels that are present in DRG neurons and that allow the entry of QX-314 and other large molecules. Compound 1 was applied at 100 micromolar together with 300 nM capsaicin for 30 minutes and then washed off. After this pretreatment, the sodium current from TRPV1 expressing neurons was measured by patch clamp electrophysiology. The sodium currents in DRG neurons treated with 100 micromolar of compound 1 were considerably smaller than those from neurons treated with capsaicin alone (FIG. 5 and FIG. 6), indicating that the compound did, indeed permeate into TRPV1 expressing DRG neurons and blocked endogenous Naᵥ currents. Also, the blocking effect of Compound 1 applied at 100 micromolar together with capsaicin on DRG Naᵥ currents was greater than that of QX-314 applied at 100 micromolar together with capsaicin (FIG. 6), consistent with the greater potency of compound 1 compared with QX-314 for blocking Nav1.7 channels when applied intracellularly.

### Example 7—Efficacy of Inhaled Compounds 1 and 2 on Cough Reflex in Antigen Induced Pulmonary Inflammation in Ovalbumin Sensitized Guinea Pig

This example demonstrates that compound 1 showed 81% inhibition and compound 2 showed 65% inhibition of citric acid induced cough in ovalbumin sensitized guinea pigs compared to a control group dosed with the vehicle.

Procedure:

Dunkin Hartley Guinea pigs were used in this study (male and female). On Day 0 all animals were sensitized with an intraperitoneal and subcutaneous injection of 50 mg chicken egg albumin (ovalbumin). Animals were administered 1 mL of a 50 mg/mL ovalbumin in 0.9% w/v saline solution via the intraperitoneal route and 0.5 mL of the same solution into 2 separate subcutaneous sites (1 mL in total divided between the left and right flank). All animals were administered a single intraperitoneal dose of pyrilamine (15 mg/kg) at a dose volume of 1 mL/kg approximately 30 minutes prior to ovalbumin challenge on Day 14 to inhibit histamine-induced bronchospasm. On Day 14, animals were challenged with aerosolised ovalbumin in 0.9% w/v saline (3 mg/mL) for 15 min.

Animals were placed in groups in an acrylic box. Then 8 mL of ovalbumin in saline was placed in each of two jet nebulisers (Sidestream®). Compressed air was passed through each nebuliser at approximately 6 L/min and the output of the nebulisers passed into the box containing the animals. On Day 15, approximately 24 hours after the inflammatory challenge with ovalbumin, animals were dosed with either vehicle, compound 1, or compound 2 by inhalation. One hour following the end of vehicle, compound 1, or compound 2 administration on Day 15, animals were placed into a whole body plethysmograph connected to a Buxco Finepointe system. All animals were then exposed to nebulized 400 mM citric acid for 7 minutes via an Aeroneb® nebuliser.

Cough counts and respiratory parameters (respiratory rate, tidal volume, minute volume and Penh) were recorded throughout the exposure period and for 10 minutes following the end of nebulization period.

Results:

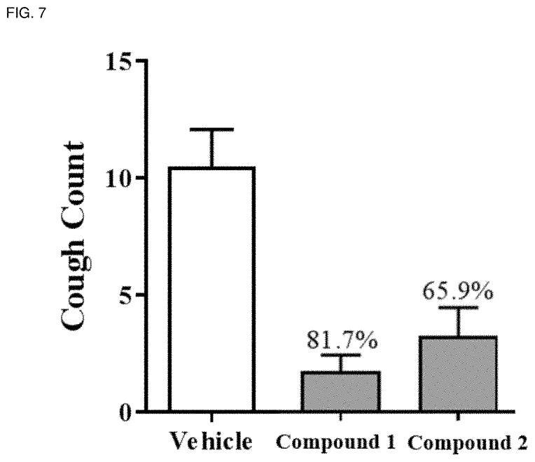

As shown in FIG. 7, compound 1 showed 81% inhibition and compound 2 showed 65% inhibition of cough in ovalbumin sensitized guinea pigs. The total cough count of each of the animals in the control group and the group dosed with vehicle, compound 1, or compound 2 are shown in Tables 2 and 3 below. The allergic airway inflammation activated large pore channels in nociceptors innervating the airways allowing entry of the charged sodium channel blockers into the sensory neurons. This silenced the neurons such that their activation by the citric acid challenge was substantially reduced as revealed by the inhibition of cough. Inhaled administration of the charged sodium channel blockers in non-inflamed guinea pigs does not block cough produced by a citric acid challenge.

TABLE 2
Cough Count-Vehicle

| Animal | Cough |
|---|---|
| No./Gender | Count |
| 1M | 14 |
| 2M | 5 |
| 3M | 9 |
| 4M | 12 |
| 5M | 19 |
| 6M | 6 |
| 7F | 9 |
| 8F | 15 |
| 9F | 10 |
| 10F | 9 |
| 11F | 18 |
| 12F | 0 |

TABLE 3
Cough Count-Compounds 1 and 2

| Animal | | Cough | Animal | | Cough |
|---|---|---|---|---|---|
| No./Gender | Group | Count | No./Gender | Group | Count |
| 37M | Compound 1 | 0 | 25M | Compound 2 | 6 |
| 38M | Compound 1 | 0 | 26M | Compound 2 | 0 |
| 39M | Compound 1 | 0 | 27M | Compound 2 | 1 |
| 40M | Compound 1 | 0 | 28M | Compound 2 | 0 |
| 41M | Compound 1 | 4 | 29M | Compound 2 | 8 |
| 42M | Compound 1 | 0 | 30M | Compound 2 | 0 |
| 43F | Compound 1 | 0 | 31F | Compound 2 | 2 |
| 44F | Compound 1 | 4 | 32F | Compound 2 | 13 |
| 45F | Compound 1 | 2 | 33F | Compound 2 | 11 |
| 46F | Compound 1 | 7 | 34F | Compound 2 | 0 |
| 47F | Compound 1 | 6 | 35F | Compound 2 | 2 |
| 48F | Compound 1 | 0 | 36F | Compound 2 | 0 |

### OTHER EMBODIMENTS

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

All publications mentioned in this specification are herein incorporated by reference to the same extent as if each independent publication was specifically and individually incorporated by reference.

1. A compound having the structure: 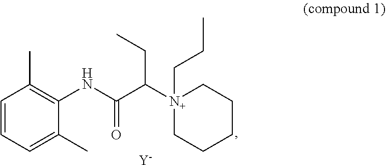
wherein Y⁻ is absent or a counterion.

2-14. (canceled)

15. A pharmaceutical composition comprising any one of the compound of claim 1 and a pharmaceutically acceptable excipient.

16. The pharmaceutical composition of claim 15, wherein the composition is formulated for oral, intravenous, intramuscular, rectal, cutaneous, subcutaneous, topical, transdermal, sublingual, nasal, inhalation, vaginal, intrathecal, epidural, or ocular administration.

17. A method for treating pain in a patient, the method comprising administering to the patient the compound of claim 1.

18. The method of claim 17, wherein the pain is selected from the group consisting of neuropathic pain, inflammatory pain, nociceptive pain, pain due to infections, pain due to trauma, surgical pain, and procedural pain.

19. A method for treating neurogenic inflammatory disorder, or a symptom associated therewith, in a patient, the method comprising administering to the patient the compound of claim 1.

20. The method of claim 19, wherein the neurogenic inflammatory disorder is selected from the group consisting of allergic inflammation, asthma, chronic cough, conjunctivitis, rhinitis, psoriasis, inflammatory bowel disease, and interstitial cystitis, atopic dermatitis.

21. A method for treating cough in a patient, the method comprising administering to the patient the compound of claim 1.

22. The method of claim 21, wherein the cough is dry cough, wet cough, croup cough, chronic cough, acute cough, or whooping cough.

23. The method of claim 22, wherein the cough is chronic cough.

24. The method of claim 22, wherein the cough is acute cough.

25-26. (canceled)
